(19) United States
(12) Patent Application Publication
(10) Pub. No.: US 2019/0046532 A1
(43) Pub. Date: Feb. 14, 2019

(54) PSYCHIATRIC TREATMENT FOR PATIENTS WITH GENE POLYMORPHISMS

(71) Applicant: INDIVIOR UK LIMITED, Slough, Berkshire (GB)

(72) Inventors: Zacharoula Konsoula, Richmond, VA (US); John Parick Jones, Richmond, VA (US); Philip Twumasi-Ankrah, Richmond, VA (US); Christian Heidbreder, Richmond, VA (US); Azmi Nasser, Richmond, VA (US)

(21) Appl. No.: 15/763,777

(22) PCT Filed: Sep. 29, 2016
(86) PCT No.: PCT/US2016/054498

§ 371 Date: Mar. 27, 2018

Related U.S. Application Data
(60) Provisional application No. 62/235,400, filed on Sep. 30, 2015.
(60) Provisional application No. 62/302,445, filed on Mar. 2, 2016.
(60) Provisional application No. 62/366,768, filed on Jul. 26, 2016.

(51) Int. Cl. A61K 31/519; A61K 9/50; A61P 25/18

(52) U.S. Cl. CPC A61K 31/519; A61P 25/18; A61K 9/5031

(57) ABSTRACT

The disclosure provides methods for treating psychiatric diseases, such as schizophrenia, in patients with certain gene polymorphisms using antipsychotic drugs, such as risperidone and paliperidone.

### RELATED APPLICATIONS

This application claims priority to U.S. Application No. 62/366,768 filed Jul. 26, 2016; to U.S. Application No. 62/302,445 filed Mar. 2, 2016; and to U.S. Application No. 62/235,400 filed Sep. 30, 2015; the disclosures of each of which are incorporated by reference herein in their entirety.

### FIELD

The disclosure provides methods for treating psychiatric diseases, such as schizophrenia, in patients with certain gene polymorphisms using antipsychotic drugs, such as risperidone and paliperidone.

### BACKGROUND

Schizophrenia is a severe and persistent debilitating psychiatric illness that is generally associated with considerable morbidity and extreme disability. Due to the severity of this disorder, especially the negative impact of a psychotic episode on a human, and the diminishing recovery after each psychotic episode, there is a need to more conclusively identify individuals who have or are at risk of developing schizophrenia, for example, to confirm clinical diagnoses, to allow for prophylactic therapies, to determine appropriate therapies based on their genotypic subtype, and to provide genetic counseling for prospective parents with a history of the disorder.

Various genes and chromosomes have been implicated in etiology of schizophrenia. Whole genome scans for genes involved in schizophrenia and related schizophrenia-spectrum disorders (including schizotypal personality disorder and schizoaffective disorder) have implicated numerous autosomes as having a role in the genetic etiology of schizophrenia and related schizophrenia-spectrum disorders. Generally, these linkage scans are too low in resolution to identify specific genes, but increasingly, transmission disequilibrium (TDT, family-based association) and Case/Control association studies have evaluated a number of positional candidate genes with a good measure of success.

There is a need in the art to identify whether schizophrenic patients with certain gene polymorphisms and/or genotypes would benefit from treatment with antipsychotic drugs, in order to provide therapy to patients who would receive the most benefit from certain medications. The disclosure is directed to this, as well as other, important ends.

### SUMMARY

The disclosure provides methods for treating psychiatric diseases in humans having a polymorphism in the MC4R gene, a polymorphism in the HTR2C gene, a polymorphism in the HTR2A gene, or a combination thereof, by administering therapeutically effective amounts of antipsychotic drugs to treat the psychiatric diseases. The human may have a CC genotype, a TT genotype, or a CT/TT genotype. The psychiatric disease may be schizophrenia. The antipsychotic drug may be selected from the group consisting of risperidone, paliperidone, clozapine, loxapine, olanzapine, thioridazine, perphenazine, aripiprazole, iloperidone, ziprasidone, lurasidone, molindone, asenapine, mesoridazine, quetiapine, trifluoperazine, or a pharmaceutically acceptable salt of any of the foregoing.

The disclosure provides methods for treating psychiatric diseases in humans having a single nucleotide polymorphism in the MC4R gene, a single nucleotide polymorphism in the HTR2C gene, a single nucleotide polymorphism in the HTR2A gene, or a combination thereof, by administering therapeutically effective amounts of antipsychotic drugs to treat the psychiatric diseases. The human may have a CC genotype, a TT genotype, or a CT/TT genotype. The psychiatric disease may be schizophrenia. The antipsychotic drug may be selected from the group consisting of risperidone, paliperidone, clozapine, loxapine, olanzapine, thioridazine, perphenazine, aripiprazole, iloperidone, ziprasidone, lurasidone, molindone, asenapine, mesoridazine, quetiapine, tritluoperazine, or a pharmaceutically acceptable salt of any of the foregoing.

The disclosure provides methods for treating psychiatric diseases in humans having an rs17782313 single nucleotide polymorphism in the MC4R gene, an rs3813929 single nucleotide polymorphism in the HTR2C gene, an rs6313 single nucleotide polymorphism in the HTR2A gene, or a combination thereof, by administering therapeutically effective amounts of antipsychotic drugs to treat the psychiatric diseases. The human may have a CC genotype, a TT genotype, or a CT/TT genotype. The psychiatric disease may be schizophrenia. The antipsychotic drug may be selected from the group consisting of risperidone, paliperidone, clozapine, loxapine, olanzapine, thioridazine, perphenazine, aripiprazole, iloperidone, ziprasidone, lurasidone, molindone, asenapine, mesoridazine, quetiapine, trifluoperazine, or a pharmaceutically acceptable salt of any of the foregoing.

The disclosure provides methods for treating schizophrenia in humans in need thereof by administering a therapeutically effective amount of risperidone or a pharmaceutically acceptable salt thereof to the human to treat the schizophrenia, wherein the human has a CC/TT genotype and an rs17782313 single nucleotide polymorphism in the MC4R gene.

The disclosure provides methods for treating schizophrenia in humans in need thereof by administering a therapeutically effective amount of risperidone or a pharmaceutically acceptable salt thereof to the human to treat the schizophrenia, wherein the human has a CC genotype and an rs3813929 single nucleotide polymorphism in the HTR2C gene.

The disclosure provides methods for treating schizophrenia in humans in need thereof by administering a therapeutically effective amount of risperidone or a pharmaceutically acceptable salt thereof to the human to treat the schizophrenia, wherein the human has a CC genotype or a CC/TT genotype, and an rs6313 single nucleotide polymorphism in the HTR2A gene.

The disclosure provides methods for treating psychiatric diseases in humans in need thereof by the steps of (i) obtaining a blood sample from the human; (ii) testing the sample for a single nucleotide polymorphism in the MC4R gene, a single nucleotide polymorphism in the HTR2C gene, a single nucleotide polymorphism in the HTR2A gene, or a combination thereof; and (iii) administering therapeutically effective amounts of antipsychotic drugs to treat the psychiatric diseases when the human tests positive for a single nucleotide polymorphism in the MC4R gene, a single nucleotide polymorphism in the HTR2C gene, a single nucleotide polymorphism in the HTR2A gene, or a combination thereof. The human may have a CC genotype, a TT genotype, or a CT/TT genotype. The single nucleotide polymorphism in the HTR2C gene may be rs3813929. The single nucleotide polymorphism in the HTR2A gene may be rs6313. The single nucleotide polymorphism in the MC4R gene may be rs17782313. The psychiatric disease may be schizophrenia. The antipsychotic drug may be selected from the group consisting of risperidone, paliperidone, clozapine, loxapine, olanzapine, thioridazine, perphenazine, aripiprazole, iloperidone, ziprasidone, lurasidone, molindone, asenapine, mesoridazine, quetiapine, trifluoperazine, or a pharmaceutically acceptable salt of any of the foregoing.

The disclosure provides methods for treating psychiatric diseases in humans having a CC genotype, a TT genotype, or a CT/TT genotype, by administering therapeutically effective amounts of antipsychotic drugs to treat the psychiatric diseases. The psychiatric disease may be schizophrenia. The antipsychotic drug may be selected from the group consisting of risperidone, paliperidone, clozapine, loxapine, olanzapine, thioridazine, perphenazine, aripiprazole, iloperidone, ziprasidone, lurasidone, molindone, asenapine, mesoridazine, quetiapine, trifluoperazine, or a pharmaceutically acceptable salt of any of the foregoing.

These and other embodiments are described herein.

### BRIEF DESCRIPTION OF THE FIGURES

FIGS. 1A-C shows the PANSS total score change from baseline until the conclusion of the study for humans having the MC4R (rs17782313) gene polymorphism receiving a subcutaneous injection of placebo (FIG. 1A), 120 mg of Formulation C (FIG. 1B), or 90 mg of Formulation C (FIG. 1C) at Days 1 and 29 in the 57 day study.

FIGS. 2A-C shows the CGI-S total score change from baseline until the conclusion of the study for humans having the MC4R (rs17782313) gene polymorphism receiving a subcutaneous injection of placebo (FIG. 2A), 120 mg of Formulation C (FIG. 2B), or 90 mg of Formulation C (FIG. 2C) at Days 1 and 29 in the 57 day study.

FIGS. 3A-C shows the PANSS total score change from baseline until the conclusion of the study for humans having the HTR2C gene (rs3813929) gene polymorphism receiving a subcutaneous injection of placebo (FIG. 3A), 120 mg of Formulation C (FIG. 3B), or 90 mg of Formulation C at (FIG. 3C) Days 1 and 29 in the 57 day study.

FIGS. 4A-C shows the CGI-S total score change from baseline until the conclusion of the study for humans having the HTR2C gene (rs3813929) gene polymorphism receiving a subcutaneous injection of placebo (FIG. 4A), 120 mg of Formulation C (FIG. 4B), or 90 mg of Formulation C (FIG. 4C) at Days 1 and 29 in the 57 day study.

FIGS. 5A-C shows the PANSS total score change from baseline until the conclusion of the study for humans having the HTR2A (rs6313) gene polymorphism receiving a subcutaneous injection of placebo (FIG. 5A), 120 mg of Formulation C (FIG. 5B), or 90 mg of Formulation C (FIG. 5C) at Days 1 and 29 in the 57 day study.

FIGS. 6A-C shows the CGI-S total score change from baseline until the conclusion of the study for humans having the HTR2A (rs6313) gene polymorphism receiving a subcutaneous injection of placebo (FIG. 6A), 120 mg of Formulation C (FIG. 6B), or 90 mg of Formulation C (FIG. 6C) at Days 1 and 29 in the 57 day study.

FIG. 7 shows the structural PK model for Formulation C that was used for the analysis.

FIG. 8 shows the sample window-based mean plasma concentration of risperidone and 9-OH-risperidone vs. time. FIG. 8A shows the mean window-based mean plasma concentration for risperidone over time, wherein the lower line represents the 90 mg dose of Formulation C, and the upper line represents the 120 mg dose of Formulation C. FIG. 8B shows the mean window-based mean plasma concentration for 9-OH-risperidone over time, wherein the lower line represents the 90 mg dose of Formulation C, and the upper line represents the 120 mg dose of Formulation C.

FIG. 9 shows the pcVPC for the covariate PK model on log scale. The three lines indicated the 5th, 50th (median) and 95th percentiles of the observed data, whereas the black lines (i.e., the dotted lines and the 3rd solid line from the top of the page) represent these percentiles for the model-simulated data. The shaded regions represent the 95% confidence intervals of the simulated percentiles. FIG. 9A shows the pcVPC for the covariate PK model for risperidone, while FIG. 9B shows the pcVPC for the covariate PK model for 9-OH-risperidone.

FIG. 10 shows a comparison of mean PANSS Score across the three treatment groups through the study duration. The upper line represents the placebo group, the middle line represents the 90 mg dose of Formulation C group, and the lower line represents the 120 mg dose of Formulation C group.

FIG. 11 shows the pcVPC for final active moiety exposure—PANSS model. The solid lines indicated the 5th, 50th (median) and 95th percentiles of the observed data whereas the black lines (i.e., the dotted lines and the second solid line from the top) represent these percentiles for the model-simulated data. The shaded regions represent the 95% confidence intervals of the simulated percentiles.

FIG. 12 shows the distribution of consolidated CGI-S scores by dose. FIG. 12A shows the placebo group where Day 1 had CGI-S scores of 4 (lower block), 5 (middle block), and 6 (upper block); and Days 15, 29, 43, and 57 had CGI-S scores of 1-3 (lower block), 4 (lower middle block), 5 (upper middle block), and 6 (upper block). FIG. 12B shows the 90 mg Formulation C group where Day 1 had CGI-S scores of 4 (lower block), 5 (middle block), and 6 (upper block); and Days 15, 29, 43, and 57 had CGI-S scores of 1-3 (lower block), 4 (lower middle block), 5 (upper middle block), and 6 (upper block). FIG. 12C shows the placebo group where Day 1 had CGI-S scores of 4 (lower block), 5 (middle block), and 6 (upper block); Days 15 and 29 had CGI-S scores of 1-3 (lower block), 4 (lower middle block), 5 (upper middle block), and 6 (upper block); and Days 43 and 57 had CGI-S scores of 1-3 (lower block), 4 (middle block), and 5 (upper block).

FIG. 13A-D show the visual predictive checks for CGI-S vs. time for marked (FIG. 13A), severe (FIG. 13B), mild (FIG. 13C), and moderate (FIG. 13D). The circles are the observed proportions, whereas the lines are the medians and the 5th and 95th percentiles of the simulation that is also shown by the shaded area.

### DETAILED DESCRIPTION

“Polymorphism” refers to a DNA sequence variation which provides for the presence of two or more distinct phenotypes in a population due to the expression of different alleles for a given gene A polymorphism can include, for example, one or more nucleotide insertions in a DNA sequence, one or more nucleotide deletions in a DNA sequence, one or more nucleotide variations in a DNA sequence, or combinations thereof.

A “single nucleotide polymorphism,” or “SNP,” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein (e.g., rs6313, rs17782313, rs3813929) refers to the official Reference SNP (rs) ID identification tag as assigned to each unique SNP by the National Center for Biotechnological Information (NCBI) or identifies the residue change associated with the identified polymorphism. SNP genotyping arrays have become an important tool for cohort identification and stratification, phenotype-genotype association studies, discovery of disease markers, prediction of molecular phenotypes, and clinical decision support.

“HTR2A” or “HTR2A gene” refers to 5-hydroxytryptamine (serotonin) receptor 2A.

“HTR2C′” or “HTR2C gene” refers to 5-hydroxytryptamine (serotonin) receptor 2C.

“MC4R” or “MC4R gene” refers to the melanocortin 4 receptor.

“rs6313” refers to the rs6313 single nucleotide polymorphism in the HTR2A gene. rs6313 is also known as T102C or C102T.

“rs17782313” refers to the rs17782313 single nucleotide polymorphism in the MC4R gene.

“rs3813929” refers to the rs3813929 single nucleotide polymorphism in the HTR2C gene. rs3813929 is also known as −759C/T.

“Psychiatric disease” refers to any disease in the Diagnostic and Statistical Manual of Mental Disorders, Fifth Edition (DSM-5), the disclosure of which is incorporated by reference herein. In embodiments, the psychiatric disease is schizophrenia. In embodiments, the psychiatric disease is bipolar disorder. In embodiments, the psychiatric disease is bipolar mania. In embodiments, the psychiatric disease is autism. In embodiments, the psychiatric disease is anxiety disorder, social phobia, attention-deficit hyperactivity disorder, depression, an eating disorder, insomnia, obsessive-compulsive disorder, personality disorder, post-traumatic stress disorder, substance abuse, or Tourette's syndrome.

“Schizophrenia” is a psychiatric disease characterized by, e.g., delusions, hallucinations, disorganized speech, grossly disorganized or cationic behavior, and negative symptoms. Schizophrenia can be acute schizophrenia. Schizophrenia can be further classified as paranoid schizophrenia, disorganized schizophrenia, undifferentiated schizophrenia, catatonic schizophrenia, or residual schizophrenia. Diagnostic criteria for schizophrenia are set forth in the DSM-5, the disclosure of which is incorporated by reference herein.

“Therapeutically effective amount” refers to an amount of the drug sufficient to contribute to the treatment or reduction of a symptom or symptoms of a psychiatric disease.

“Administering” refers to oral administration, administration as a suppository, topical, intravenous, parenteral, intraperitoneal, intramuscular, intrathecal, intracranial, intranasal or subcutaneous administration, or the implantation of a slow-release device (e.g., a solid polymeric biodegradable device, a mini-osmotic pump) to a subject. Administration is by any route, including parenteral and transmucosal (e.g., buccal, sublingual, palatal, gingival, nasal, or transdermal). Parenteral administration includes, e.g., intravenous, intramuscular, intra-arteriole, intradermal, subcutaneous, intraperitoneal, intraventricular, and intracranial. In embodiments, parenteral administration is subcutaneous administration. Other modes of delivery include, but are not limited to, the use of microsphere formulations, liposomal formulations, intravenous infusion, transdermal patches, etc. The compositions of the present invention can be delivered by transdermally, by a topical route, formulated as applicator sticks, solutions, suspensions, emulsions, gels, creams, ointments, pastes, jellies, paints, powders, and aerosols. Oral preparations include tablets, pills, powder, capsules, liquids, lozenges, cachets, gels, syrups, slurries, suspensions, etc., suitable for ingestion by the human. Solid form preparations include powders, tablets, pills, capsules, cachets, suppositories, and dispersible granules. Liquid form preparations include solutions, suspensions, and emulsions, for example, water or water/propylene glycol solutions. The compositions of the present invention may additionally include components to provide sustained release and/or comfort. Such components include high molecular weight, anionic mucomimetic polymers, gelling polysaccharides and finely-divided drug carrier substrates. The compositions of the invention can also be delivered as microspheres for slow release in the body. For example, microspheres can be administered via intradermal injection of drug-containing microspheres, which slowly release intramuscularly or subcutaneously; as biodegradable and injectable gel formulations; or as microspheres for oral administration. The compositions of the present invention can also be delivered as nanoparticles.

“Pharmaceutically acceptable excipient” refers to a compound or substance that aids the administration of an active agent to a patent and can be included in the compositions of the invention without causing a significant adverse toxicological effect on the human. Non-limiting examples of pharmaceutically acceptable excipients include water, NaCl, normal saline solutions, lactated Ringer's, normal sucrose, normal glucose, binders, fillers, disintegrants, lubricants, coatings, sweeteners, flavors, salt solutions (such as Ringer's solution), alcohols, oils, gelatins, carbohydrates such as lactose, amylose or starch, fatty acid esters, hydroxymethycellulose, polyvinyl pyrrolidine, and colors, and the like. Such preparations can be sterilized and, if desired, mixed with auxiliary agents such as lubricants, preservatives, stabilizers, wetting agents, emulsifiers, salts for influencing osmotic pressure, buffers, coloring, and/or aromatic substances and the like that do not deleteriously react with the compounds of the invention. One of skill in the art will recognize that other pharmaceutical excipients are useful. In embodiments of the methods described herein, the pharmaceutical compositions will comprise an antipsychotic drug (e.g., risperidone, paliperidone) and at least one pharmaceutically acceptable excipient.

“Pharmaceutically acceptable salt” refers to salts of the active compounds that are prepared with relatively nontoxic acids or bases, depending on the particular substituents found on the compounds described herein. When compounds of the invention contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, fumaric, lactic, mandelic, phthalic, benzenesulfonic, p-tolylsulfonic, citric, tartaric, methanesulfonic, and the like.

“Treating” or “treatment” refers to any indicia of success in the treatment or amelioration of a psychiatric disease, including any objective or subjective parameter such as abatement; remission; diminishing of symptoms or making the condition more tolerable to the human; slowing in the rate of degeneration or decline; making the final point of degeneration less debilitating; improving a human's physical or mental well-being. The success in the treatment or amelioration of symptoms can be based on objective or subjective parameters; including the results of a physical examination, neuropsychiatric exams, and/or a psychiatric evaluation.

“Month” means 28 days to 31 days. In one embodiment, a month is 28 days, 29 days, 30 days, or 31 days. In one embodiment, a month is 28 days. In one embodiment, a month is 30 days. In one embodiment, a month is 31 days.

The disclosure provides methods for treating psychiatric diseases, such as schizophrenia, in humans having a polymorphism in the MC4R gene, a polymorphism in the HTR2C gene, a polymorphism in the HTR2A gene, or a combination thereof, by administering to the humans therapeutically effective amounts of risperidone or a pharmaceutically acceptable salt thereof to treat the psychiatric disease. The human may have a CC genotype, a TT genotype, or a CT/TT genotype.

The disclosure provides methods for treating psychiatric diseases, such as schizophrenia, in humans having a single nucleotide polymorphism in the MC4R gene, a single nucleotide polymorphism in the HTR2C gene, a single nucleotide polymorphism in the HTR2A gene, or a combination thereof, by administering to the humans therapeutically effective amounts of risperidone or a pharmaceutically acceptable salt thereof to treat the psychiatric disease. The human may have a CC genotype, a TT genotype, or a CT/TT genotype. The single nucleotide polymorphism in the HTR2C gene may be rs3813929. The single nucleotide polymorphism in the HTR2A gene may be rs6313. The single nucleotide polymorphism in the MC4R gene may be rs17782313.

The disclosure provides methods for treating psychiatric diseases, such as schizophrenia, in humans having a CC genotype, a TT genotype, or a CT/TT genotype by administering to the humans therapeutically effective amounts of risperidone or a pharmaceutically acceptable salt thereof to treat the psychiatric disease. The human may have a polymorphism in the MC4R gene, a polymorphism in the HTR2C gene, a polymorphism in the HTR2A gene, or a combination thereof. The human may have a single nucleotide polymorphism in the MC4R gene, a single nucleotide polymorphism in the HTR2C gene, a single nucleotide polymorphism in the HTR2A gene, or a combination thereof. The single nucleotide polymorphism in the HTR2C gene may be rs3813929. The single nucleotide polymorphism in the HTR2A gene may be rs6313. The single nucleotide polymorphism in the MC4R gene may be rs17782313.

The disclosure provides methods for treating psychiatric diseases, such as schizophrenia, in humans having a polymorphism in the MC4R gene, a polymorphism in the HTR2C gene, a polymorphism in the HTR2A gene, or a combination thereof, by administering to the humans therapeutically effective amounts of paliperidone or a pharmaceutically acceptable salt thereof to treat the psychiatric disease. The human may have a CC genotype, a TT genotype, or a CT/TT genotype.

The disclosure provides methods for treating psychiatric diseases, such as schizophrenia, in humans having a single nucleotide polymorphism in the MC4R gene, a single nucleotide polymorphism in the HTR2C gene, a single nucleotide polymorphism in the HTR2A gene, or a combination thereof, by administering to the humans therapeutically effective amounts of paliperidone or a pharmaceutically acceptable salt thereof to treat the psychiatric disease. The human may have a CC genotype, a TT genotype, or a CT/TT genotype. The single nucleotide polymorphism in the HTR2C gene may be rs3813929. The single nucleotide polymorphism in the HTR2A gene may be rs6313. The single nucleotide polymorphism in the MC4R gene may be rs17782313.

The disclosure provides methods for treating psychiatric diseases, such as schizophrenia, in humans having a CC genotype, a TT genotype, or a CT/TT genotype by administering to the humans therapeutically effective amounts of paliperidone or a pharmaceutically acceptable salt thereof to treat the psychiatric disease. The human may have a polymorphism in the MC4R gene, a polymorphism in the HTR2C gene, a polymorphism in the HTR2A gene, or a combination thereof. The human may have a single nucleotide polymorphism in the MC4R gene, a single nucleotide polymorphism in the HTR2C gene, a single nucleotide polymorphism in the HTR2A gene, or a combination thereof. The single nucleotide polymorphism in the HTR2C gene may be rs3813929. The single nucleotide polymorphism in the HTR2A gene may be rs6313. The single nucleotide polymorphism in the MC4R gene may be rs17782313.

The disclosure provides methods for treating schizophrenia in humans having a polymorphism in the MC4R gene, a polymorphism in the HTR2C gene, a polymorphism in the HTR2A gene, or a combination thereof, by administering to the humans pharmaceutical compositions comprising therapeutically effective amounts of risperidone base, a poly(lactide-co-glycolide)copolymer, and N-methyl-2-pyrrolidone, to treat the schizophrenia. The human may have a CC genotype, a TT genotype, or a CT/TT genotype. In one embodiment, the pharmaceutical composition is Formulation A. In one embodiment, the pharmaceutical composition is Formulation B. In one embodiment, the pharmaceutical composition is Formulation C.

The disclosure provides methods for treating schizophrenia in humans having a single nucleotide polymorphism in the MC4R gene, a single nucleotide polymorphism in the HTR2C gene, a single nucleotide polymorphism in the HTR2A gene, or a combination thereof, by administering to the humans pharmaceutical compositions comprising therapeutically effective amounts of risperidone base, a poly(lactide-co-glycolide)copolymer, and N-methyl-2-pyrrolidone, to treat the schizophrenia. The human may have a CC genotype, a TT genotype, or a CT/TT genotype. The single nucleotide polymorphism in the HTR2C gene may be rs3813929. The single nucleotide polymorphism in the HTR2A gene may be rs6313. The single nucleotide polymorphism in the MC4R gene may be rs17782313. In one embodiment, the pharmaceutical composition is Formulation A. In one embodiment, the pharmaceutical composition is Formulation B. In one embodiment, the pharmaceutical composition is Formulation C.

The disclosure provides methods for treating schizophrenia in humans with a CT/TT genotype and a single nucleotide polymorphism in the MC4R gene by administering to the humans therapeutically effective amounts of risperidone or a pharmaceutically acceptable salt thereof to treat the schizophrenia. The single nucleotide polymorphism in the MC4R gene may be rs17782313.

The disclosure provides methods for treating schizophrenia in humans with a CT/TT genotype and a single nucleotide polymorphism in the MC4R gene by administering to the humans therapeutically effective amounts of paliperidone or a pharmaceutically acceptable salt thereof to treat the schizophrenia. The single nucleotide polymorphism in the MC4R gene may be rs17782313.

The disclosure provides methods for treating schizophrenia in humans with the CC genotype and a single nucleotide polymorphism in the HTR2C gene by administering to the humans therapeutically effective amounts of risperidone or a pharmaceutically acceptable salt thereof to treat the schizophrenia. The single nucleotide polymorphism in the HTR2C gene may be rs3813929.

The disclosure provides methods for treating schizophrenia in humans with the CC genotype and a single nucleotide polymorphism in the HTR2C gene by administering to the humans therapeutically effective amounts of paliperidone or a pharmaceutically acceptable salt thereof to treat the schizophrenia. The single nucleotide polymorphism in the HTR2C gene may be rs3813929.

The disclosure provides methods for treating schizophrenia in humans with the CC genotype and a single nucleotide polymorphism in the HTR2A gene by administering to the humans therapeutically effective amounts of risperidone or a pharmaceutically acceptable salt thereof to treat the schizophrenia. The single nucleotide polymorphism in the HTR2A gene may be rs6313.

The disclosure provides methods for treating schizophrenia in humans with the CC genotype and a single nucleotide polymorphism in the HTR2A gene by administering to the humans therapeutically effective amounts of paliperidone or a pharmaceutically acceptable salt thereof to treat the schizophrenia. The single nucleotide polymorphism in the HTR2A gene may be rs6313.

The disclosure provides methods for treating schizophrenia in humans with the CT/TT genotype in the HTR2A single nucleotide polymorphism by administering to the humans therapeutically effective amounts of risperidone or a pharmaceutically acceptable salt to treat the schizophrenia. The single nucleotide polymorphism in the HTR2A gene may be rs6313.

The disclosure provides methods for treating schizophrenia in humans with the CT/TT genotype in the HTR2A single nucleotide polymorphism by administering to the humans therapeutically effective amounts of paliperidone or a pharmaceutically acceptable salt thereof to treat the schizophrenia. The single nucleotide polymorphism in the HTR2A gene may be rs6313.

The disclosure provides methods for treating psychiatric diseases in humans in need thereof by the steps of (i) obtaining a blood sample from the human; (ii) testing the sample for a polymorphism in the MC4R gene; and (iii) administering therapeutically effective amounts of risperidone or a pharmaceutically acceptable salt thereof or paliperidone or a pharmaceutically acceptable salt thereof to treat the psychiatric diseases when the human tests positive for the polymorphism in the MC4R gene. The human may have a CC genotype, a TT genotype, or a CT/TT genotype.

The disclosure provides methods for treating psychiatric diseases in humans in need thereof by the steps of (i) obtaining a blood sample from the human; (ii) testing the sample for a single nucleotide polymorphism in the MC4R gene; and (iii) administering therapeutically effective amounts of risperidone or a pharmaceutically acceptable salt thereof to treat the psychiatric diseases when the human tests positive for a single nucleotide polymorphism in the MC4R gene. The human may have a CC genotype, a TT genotype, or a CT/TT genotype. The single nucleotide polymorphism in the MC4R gene may be rs17782313. The psychiatric disease may be schizophrenia. In embodiments, the humans have a CC/TT genotype and an rs17782313 single nucleotide polymorphism in the MC4R gene.

The disclosure provides methods for treating psychiatric diseases in humans in need thereof by the steps of (i) obtaining a blood sample from the human; (ii) testing the sample for a single nucleotide polymorphism in the MC4R gene; and (iii) administering therapeutically effective amounts of paliperidone or a pharmaceutically acceptable salt thereof to treat the psychiatric diseases when the human tests positive for a single nucleotide polymorphism in the MC4R gene. The human may have a CC genotype, a TT genotype, or a CT/TT genotype. The single nucleotide polymorphism in the MC4R gene may be rs17782313. The psychiatric disease may be schizophrenia. In embodiments, the humans have a CC/TT genotype and an rs17782313 single nucleotide polymorphism in the MC4R gene.

The disclosure provides methods for treating psychiatric diseases in humans in need thereof by the steps of (i) obtaining a blood sample from the human; (ii) testing the sample for a polymorphism in the HTR2C gene; and (iii) administering therapeutically effective amounts of risperidone or a pharmaceutically acceptable salt thereof or paliperidone or a pharmaceutically acceptable salt thereof to treat the psychiatric diseases when the human tests positive for the polymorphism in the HTR2C gene. The human may have a CC genotype, a TT genotype, or a CT/TT genotype.

The disclosure provides methods for treating psychiatric diseases in humans in need thereof by the steps of (i) obtaining a blood sample from the human; (ii) testing the sample for a single nucleotide polymorphism in the HTR2C gene; and (iii) administering therapeutically effective amounts of risperidone or a pharmaceutically acceptable salt thereof to treat the psychiatric diseases when the human tests positive for a single nucleotide polymorphism in the HTR2C gene. The human may have a CC genotype, a TT genotype, or a CT/TT genotype. The single nucleotide polymorphism in the HTR2C gene may be rs3813929. The psychiatric disease may be schizophrenia. In embodiments, the humans have a CC genotype and an rs3813929 single nucleotide polymorphism in the HTR2C gene.

The disclosure provides methods for treating psychiatric diseases in humans in need thereof by the steps of (i) obtaining a blood sample from the human; (ii) testing the sample for a single nucleotide polymorphism in the HTR2C gene; and (iii) administering therapeutically effective amounts of paliperidone or a pharmaceutically acceptable salt thereof to treat the psychiatric diseases when the human tests positive for a single nucleotide polymorphism in the HTR2C gene. The human may have a CC genotype, a TT genotype, or a CT/TT genotype. The single nucleotide polymorphism in the HTR2C gene may be rs3813929. The psychiatric disease may be schizophrenia. In embodiments, the humans have a CC genotype and an rs3813929 single nucleotide polymorphism in the HTR2C gene.

The disclosure provides methods for treating psychiatric diseases in humans in need thereof by the steps of (i) obtaining a blood sample from the human; (ii) testing the sample for a polymorphism in the HTR2A gene; and (iii) administering therapeutically effective amounts risperidone or a pharmaceutically acceptable salt thereof or paliperidone or a pharmaceutically acceptable salt thereof to treat the psychiatric diseases when the human tests positive for a polymorphism in the HTR2A gene. The human may have a CC genotype, a TT genotype, or a CT/TT genotype.

The disclosure provides methods for treating psychiatric diseases in humans in need thereof by the steps of (i) obtaining a blood sample from the human; (ii) testing the sample for a single nucleotide polymorphism in the HTR2A gene; and (iii) administering therapeutically effective amounts risperidone or a pharmaceutically acceptable salt thereof to treat the psychiatric diseases when the human tests positive for a single nucleotide polymorphism in the HTR2A gene. The human may have a CC genotype, a TT genotype, or a CT/TT genotype. The single nucleotide polymorphism in the HTR2A gene may be rs6313. The psychiatric disease may be schizophrenia. In embodiments, the humans have a CC genotype and an rs6313 single nucleotide polymorphism in the HTR2A gene. In embodiments, the humans have a CC/TT genotype and an rs6313 single nucleotide polymorphism in the HTR2A gene.

The disclosure provides methods for treating psychiatric diseases in humans in need thereof by the steps of (i) obtaining a blood sample from the human; (ii) testing the sample for a single nucleotide polymorphism in the HTR2A gene; and (iii) administering therapeutically effective amounts paliperidone or a pharmaceutically acceptable salt thereof to treat the psychiatric diseases when the human tests positive for a single nucleotide polymorphism in the HTR2A gene. The human may have a CC genotype, a TT genotype, or a CT/TT genotype. The single nucleotide polymorphism in the HTR2A gene may be rs6313. The psychiatric disease may be schizophrenia. In embodiments, the humans have a CC genotype and an rs6313 single nucleotide polymorphism in the HTR2A gene. In embodiments, the humans have a CC/TT genotype and an rs6313 single nucleotide polymorphism in the HTR2A gene.

In embodiments, the pharmaceutical composition may contain risperidone in the form of the free base or a pharmaceutically acceptable salt. In embodiments, risperidone is in the form of the free base. In embodiments, risperidone is in the form of a pharmaceutically acceptable salt. In embodiments, the pharmaceutical composition may contain prodrugs of risperidone or metabolites of risperidone. In embodiments, the risperidone has a particle size distribution such that at least 75% of the particles have a size from about 1 micron to about 250 microns. In embodiments, the risperidone has a particle size distribution such that at least 75% of the particles have a size from about 1 micron to about 40 microns. In embodiments, the risperidone has a particle size distribution such that at least 75% of the particles have a size from about 50 microns to about 150 microns. The particle size distribution may be determined by methods known in the art, such as laser light scattering/diffraction method following the Fraunhofer diffraction theory.

In embodiments, the pharmaceutical composition may contain paliperidone in the form of the free base or a pharmaceutically acceptable salt. In embodiments, paliperidone is in the form of the free base. In embodiments, paliperidone is in the form of a pharmaceutically acceptable salt. In embodiments, the pharmaceutical composition may contain prodrugs of paliperidone or metabolites of paliperidone. Paliperidone is also known as 9-hydroxy-risperidone, an active metabolite of risperidone. In embodiments, the paliperidone has a particle size distribution such that at least 75% of the particles have a size from about 1 micron to about 250 microns. In embodiments, the paliperidone has a particle size distribution such that at least 75% of the particles have a size from about 1 micron to about 40 microns. In embodiments, the paliperidone has a particle size distribution such that at least 75% of the particles have a size from about 50 microns to about 150 microns. The particle size distribution may be determined by methods known in the art, such as laser light scattering/diffraction following the Fraunhofer diffraction theory.

In embodiments, the pharmaceutical composition containing risperidone can be RISPERDAL® CONSTA® (risperidone intramuscular injection by Janssen Pharmaceuticals) or a generic equivalent thereof. Such compositions and methods of making them are described in U.S. Pat. No. 5,688,801, U.S. Pat. No. 5,792,477, U.S. Pat. No. 5,916,598, U.S. Pat. No. 6,194,006, and U.S. Pat. No. 6,667,061, the disclosures of which are incorporated by reference herein in their entirety.

In embodiments, the pharmaceutical composition containing risperidone can be an oral solution, an oral suspension, an oral capsule, or an oral tablet, such as RISPERDAL® (risperidone oral solution or oral tablet, both by Janssen Pharmaceuticals) or a generic equivalent thereof. Such compositions and methods of making them are described in U.S. Pat. No. 5,648,093 and U.S. Pat. No. 6,224,905, the disclosures of which are incorporated by reference herein in their entirety.

In embodiments, the pharmaceutical composition can be a solid implantable composition containing risperidone and a biodegradable polymer. In embodiments, the solid implantable composition contains risperidone and a polymer selected from the group consisting of a polylactic acid, a polyglycolic acid, and a combination thereof. In embodiments, the solid implantable composition contains risperidone and a poly(lactide-co-glycolide) copolymer. In embodiments, the solid implantable composition is administered to the patient by a surgical implant technique. Such solid implantable pharmaceutical compositions containing risperidone are described in U.S. Pat. No. 9,439,905, U.S. Pat. No. 8,802,127, U.S. Pat. No. 8,741,327, and U.S. Pat. No. 8,221,778, the disclosures of which are incorporated by reference herein in their entirety.

In embodiments, the pharmaceutical composition can be an injectable depot formulation containing risperidone in the form of a base or a pharmaceutically acceptable salt, a poly(lactide-co-glycolide) copolymer having a number average molecular weight from about 1000 Daltons to about 5000 Daltons, and an organic solvent selected from the group consisting of benzyl alcohol, benzyl benzoate, ethyl benzoate, N-methyl-2-pyrrolidone, or a combination of two thereof; wherein the risperidone has a particle size distribution such that at least 75% of the particles have a size ranging from about 1 micron to about 40 microns (or less than 38 microns) when measured by a laser light scattering/diffraction technique, such as the Fraunhofer diffraction theory. Such compositions are described in U.S. Pat. No. 9,044,450 and U.S. Pat. No. 8,852,638, the disclosures of which are incorporated by reference herein in their entirety.

In embodiments, the pharmaceutical composition containing paliperidone can be an oral capsule or tablet, such as INVEGA® (paliperidone oral tablet by Janssen Pharmaceuticals) or a generic equivalent thereof. In embodiments, the pharmaceutical composition containing paliperidone can be an injectable solution or an injectable suspension, such as INVEGA® SUSTENNA® or INVEGA TRINZA™ (paliperidone intramuscular injection by Janssen Pharmaceuticals), or generic equivalents thereof. Such compositions and methods of making them are described in U.S. Pat. No. 6,077,843 and U.S. Pat. No. 6,555,544, the disclosures of which are incorporated by reference herein in their entirety.

In embodiments, the pharmaceutical composition can be a solid implantable composition containing paliperidone and a biodegradable polymer. In embodiments, the solid implantable composition contains paliperidone and a polymer selected from the group consisting of a polylactic acid, a polyglycolic acid, and a combination thereof. In embodiments, the solid implantable composition contains paliperidone and a poly(lactide-co-glycolide) copolymer. In embodiments, the solid implantable composition is administered to the patient by a surgical implant technique. Such solid implantable pharmaceutical compositions containing paliperidone are described in U.S. Pat. No. 9,439,905, U.S. Pat. No. 8,802,127, U.S. Pat. No. 8,741,327, and U.S. Pat. No. 8,221,778, the disclosures of which are incorporated by reference herein in their entirety.

In embodiments, the pharmaceutical composition can be an injectable depot formulation containing paliperidone in the form of a base or a pharmaceutically acceptable salt; a poly(lactide-co-glycolide) copolymer having a number average molecular weight from about 1000 Daltons to about 5000 Daltons, and an organic solvent selected from the group consisting of benzyl alcohol, benzyl benzoate, ethyl benzoate, N-methyl-2-pyrrolidone, or a combination of two thereof; wherein the paliperidone has a particle size distribution such that at least 75% of the particles have a size ranging from about 1 micron to about 40 microns (or less than 38 microns) when measured by a laser light scattering/diffraction technique, such as the Fraunhofer diffraction theory. Such compositions are described in U.S. Pat. No. 9,044,450 and U.S. Pat. No. 8,852,638, the disclosures of which are incorporated by reference herein in their entirety.

In embodiments, the pharmaceutical composition may contain any antipsychotic agent. Exemplary antipsychotic agents that can be used in the methods of treatment described herein include risperidone, paliperidone, clozapine, loxapine, olanzapine, thioridazine, perphenazine, aripiprazole, iloperidone, ziprasidone, lurasidone, molindone, asenapine, mesoridazine, quetiapine, trifluoperazine, or a pharmaceutically acceptable salt of any of the foregoing.

In embodiments, the pharmaceutical composition containing risperidone or paliperidone is in a form suitable for subcutaneous injection, such formulations and methods of making them are described in U.S. Pat. No. 9,180,197, the disclosure of which is incorporated by reference herein in its entirety.

In embodiments, the pharmaceutical composition containing risperidone is Formulation A, wherein Formulation A comprises about 5 wt % to about 25 wt % risperidone base; about 25 wt % to about 50 wt % of a poly(lactide-co-glycolide)copolymer; and about 35 wt % to about 60 wt % N-methyl-2-pyrrolidone. Formulation A can be prepared by processes known in the art and described, for example, in U.S. Pat. No. 9,180,197, the disclosure of which is incorporated by reference herein in its entirety. In embodiments, Formulation A is parenterally administered to a human from about once every 2 weeks to about once every 8 weeks. In embodiments, Formulation A is parenterally administered to a human from about once a month to about once every 6 weeks. In embodiments, Formulation A is parenterally administered to a human about once a month. In embodiments, parenteral administration is subcutaneous administration. In embodiments, risperidone is present in Formulation A in an amount from about 25 mg to about 200 mg; from about 50 mg to about 150 mg; from about 60 mg to about 150 mg; from about 80 mg to about 140 mg; from about 90 mg to about 120 mg. In embodiments, risperidone is present in Formulation A in an amount of about 60 mg. In embodiments, risperidone is present in Formulation A in an amount of about 90 mg. In embodiments, risperidone is present in Formulation A in an amount of about 120 mg.

In embodiments, the pharmaceutical composition containing risperidone is Formulation B, wherein Formulation B comprises about 10 wt % to about 20 wt % risperidone base; about 35 wt % to about 45 wt % of a 50:50 to 90:10 poly(lactide-co-glycolide)copolymer; and about 40 wt % to about 50 wt % N-methyl-2-pyrrolidone. Formulation B can be prepared by processes known in the art and described, for example, in U.S. Pat. No. 9,180,197, the disclosure of which is incorporated by reference herein in its entirety. In embodiments, Formulation B is parenterally administered to a human from about once every 2 weeks to about once every 8 weeks. In embodiments, Formulation B is parenterally administered to a human from about once a month to about once every 6 weeks. In embodiments, Formulation B is parenterally administered to a human about once a month. In embodiments, parenteral administration is subcutaneous administration. In embodiments, risperidone is present in Formulation B in an amount from about 25 mg to about 200 mg; from about 50 mg to about 150 mg; from about 60 mg to about 150 mg; from about 80 mg to about 140 mg; from about 90 mg to about 120 mg. In embodiments, risperidone is present in Formulation B in an amount of about 60 mg. In embodiments, risperidone is present in Formulation B in an amount of about 90 mg. In embodiments, risperidone is present in Formulation B in an amount of about 120 mg.

In embodiments, the pharmaceutical composition containing risperidone is Formulation C, wherein Formulation C comprises about 15 wt % risperidone base; about 38 wt % of an 80:20 poly(lactide-co-glycolide)copolymer having a number average molecular weight from about 20,000 Daltons to about 30,000 Daltons; and about 47 wt % N-methyl-2-pyrrolidone. Formulation C can be prepared by processes known in the art and described, for example, in U.S. Pat. No. 9,180,197, the disclosure of which is incorporated by reference herein in its entirety. In embodiments, Formulation C is parenterally administered to a human from about once every 2 weeks to about once every 8 weeks. In embodiments, Formulation C is parenterally administered to a human from about once a month to about once every 6 weeks. In embodiments, Formulation C is parenterally administered to a human about once a month. In embodiments, parenteral administration is subcutaneous administration. In embodiments, risperidone is present in Formulation C in an amount from about 25 mg to about 200 mg; from about 50 mg to about 150 mg; from about 60 mg to about 150 mg; from about 80 mg to about 140 mg; from about 90 mg to about 120 mg. In embodiments, risperidone is present in Formulation C in an amount of about 60 mg. In embodiments, risperidone is present in Formulation C in an amount of about 90 mg. In embodiments, risperidone is present in Formulation C in an amount of about 120 mg. Formulation C is also known as RBP-7000.

“Number average molecular weight” refers to the total weight of all the polymer molecules in a sample, divided by the total number of polymer molecules in a sample. Number average molecular weight can be determined by methods known in the art, such as by gel permeation chromatography/size exclusion chromatography (e.g., available from Agilent Technologies).

In embodiments, the pharmaceutical composition containing risperidone is Formulation D, wherein Formulation D comprises about 1 wt % to about 30 wt % of risperidone, a pharmaceutically acceptable salt of risperidone, paliperidone, or a pharmaceutically acceptable salt of paliperidone; about 10 wt % to about 80 wt % of a biodegradable polymer; and about 10 wt % to about 80 wt % of an organic solvent. In embodiments, the biodegradable polymer is a polylactide, a polyglycolide, a polycaprolactone, a copolymer thereof, a terpolymer thereof, or any combination thereof. In embodiments, the organic solvent is N-methyl-2-pyrrolidone, 2-pyrrolidone, acetic acid, lactic acid, methyl lactate, ethyl lactate, monomethyl succinate acid, monomethyl citric acid, glycofurol, glycerol formal, isopropylidene glycol, 2,2-dimethyl-1,3-dioxolone-4-methanol, solketal, dimethylformamide, dimethylacetamide, dimethylsulfoxide, dimethylsulfone, epsilon-caprolactone, butyrolactone, caprolactam, or a mixture of two or more thereof. Formulation D can be prepared by processes known in the art and described, for example, in U.S. Pat. No. 9,180,197, the disclosure of which is incorporated by reference herein in its entirety. In embodiments, Formulation D is parenterally administered to a human from about once every 2 weeks to about once every 8 weeks. In embodiments, Formulation D is parenterally administered to a human from about once a month to about once every 6 weeks. In embodiments, Formulation D is parenterally administered to a human about once a month. In embodiments, parenteral administration is subcutaneous administration. In embodiments, risperidone, a pharmaceutically acceptable salt of risperidone, paliperidone, or a pharmaceutically acceptable salt of paliperidone is present in Formulation D in an amount from about 1 mg to about 500 mg; from about 25 mg to about 200 mg; from about 50 mg to about 150 mg; from about 60 mg to about 150 mg; from about 80 mg to about 140 mg; from about 90 mg to about 120 mg. In embodiments, risperidone, a pharmaceutically acceptable salt of risperidone, paliperidone, or a pharmaceutically acceptable salt of paliperidone is present in Formulation D in an amount of about 60 mg. In embodiments, risperidone, a pharmaceutically acceptable salt of risperidone, paliperidone, or a pharmaceutically acceptable salt of paliperidone is present in Formulation D in an amount of about 90 mg. In embodiments, risperidone, a pharmaceutically acceptable salt of risperidone, paliperidone, or a pharmaceutically acceptable salt of paliperidone is present in Formulation D in an amount of about 120 mg.

As is well known in the art, therapeutically effective amounts for use in humans can be determined from animal models and from commercially available pharmaceutical formulations that are used to treat psychiatric diseases, such as schizophrenia, bipoloar disorder, and autism. For example, a dose for humans can be formulated to achieve a concentration that has been found to be effective in animals or humans. The dosage in humans can be adjusted by monitoring compounds effectiveness and adjusting the dosage upwards or downwards. Determination of the proper dosage for a particular situation is within the skill of the practitioner. Generally, treatment is initiated with smaller dosages which are less than the optimum dose of the compound. Thereafter, the dosage is increased by small increments until the optimum effect under circumstances is reached. In embodiments, oral dosages of antipsychotic agents, such as risperidone or paliperidone, may be from about 0.5 mg to about 10 mg, or from about 0.5 mg to about 5 mg. In embodiments, intramuscular injectable dosages of antipsychotic agents, such as paliperidone, may be from about 50 mg to about 900 mg. In embodiments, intramuscular injectable dosages of antipsychotic agents, such as risperidone, may be from about 10 mg to about 50 mg. In embodiments, subcutaneous injectable dosages of antipsychotic agents, such as risperidone, may be from about 90 mg to about 120 mg.

### EMBODIMENTS OF THE DESCRIPTION

In the following embodiments, any reference to a single embodiment encompasses a reference to each embodiment therein (e.g., a reference to Embodiment 42 encompasses a reference to Embodiment 42A and Embodiment 42B).

### Embodiment 1

A method for treating a psychiatric disease in a human having an MC4R gene polymorphism comprising administering a pharmaceutical composition comprising a therapeutically effective amount of (i) risperidone or a pharmaceutically acceptable salt thereof or (ii) paliperidone or a pharmaceutically acceptable salt thereof, to the human having the MC4R gene polymorphism to treat the psychiatric disease.

### Embodiment 2

A method for treating a psychiatric disease in a human having an HTR2C gene polymorphism comprising administering a pharmaceutical composition comprising a therapeutically effective amount of (i) risperidone or a pharmaceutically acceptable salt thereof or (ii) paliperidone or a pharmaceutically acceptable salt thereof, to the human having the HTR2C gene polymorphism to treat the psychiatric disease.

### Embodiment 3

A method for treating a psychiatric disease in a human having an HTR2A gene polymorphism comprising administering a pharmaceutical composition comprising a therapeutically effective amount of (i) risperidone or a pharmaceutically acceptable salt thereof or (ii) paliperidone or a pharmaceutically acceptable salt thereof, to the human having the HTR2A gene polymorphism to treat the psychiatric disease.

### Embodiment 4

A method for treating a psychiatric disease in a human having an MC4R gene polymorphism, an HTR2C gene polymorphism, an HTR2A gene polymorphism, or a combination thereof comprising administering a pharmaceutical composition comprising a therapeutically effective amount of (i) risperidone or a pharmaceutically acceptable salt thereof or (ii) paliperidone or a pharmaceutically acceptable salt thereof, to the human having the MC4R gene polymorphism, the HTR2C gene polymorphism, the HTR2A gene polymorphism, or the combination thereof to treat the psychiatric disease.

### Embodiment 5

The method of any one of Embodiments 1-4, wherein the human has a TT genotype.

### Embodiment 6

The method of any one of Embodiments 1-4, wherein the human has a CC genotype.

### Embodiment 7

The method of any one of Embodiments 1-4, wherein the human has a CT/TT genotype.

### Embodiment 8A

The method of any one of Embodiments 1 and 4-7, wherein the MC4R gene polymorphism is a single nucleotide polymorphism.

### Embodiment 8B

The method of Embodiment 8A, wherein the single nucleotide polymorphism is an rs17782313 single nucleotide polymorphism.

### Embodiment 9A

The method of any one of Embodiments 2 and 4-7, wherein the HTR2C gene polymorphism is a single nucleotide polymorphism.

### Embodiment 9B

The method of Embodiment 9A, wherein the single nucleotide polymorphism is an rs3813929 single nucleotide polymorphism.

### Embodiment 10A

The method of any one of Embodiments 3-7, wherein the HTR2A gene polymorphism is a single nucleotide polymorphism.

### Embodiment 10B

The method of Embodiment 10A, wherein the single nucleotide polymorphism is an rs6313 single nucleotide polymorphism.

### Embodiment 11

The method of any one of Embodiments 1-10, wherein the psychiatric disease is schizophrenia.

### Embodiment 12A

The method of Embodiment 11, wherein the schizophrenia is acute schizophrenia.

### Embodiment 12B

The method of Embodiment 11, wherein the schizophrenia is an acute psychotic state.

### Embodiment 12C

The method of Embodiment 11, wherein the schizophrenia is a relapse with acute schizophrenic symptoms.

### Embodiment 13

The method of any one of Embodiments 1-10, wherein the psychiatric disease is bipolar disorder.

### Embodiment 14

The method of any one of Embodiments 1-10, wherein the psychiatric disease is autism.

### Embodiment 15

The method of any one of Embodiments 1-14, wherein the human is an African-American human.

### Embodiment 16

The method of any one of Embodiments 1-15, wherein the pharmaceutical composition is an oral tablet or an oral capsule.

### Embodiment 17

The method of any one of Embodiments 1-15, wherein the method comprises administering the pharmaceutical composition to the human by oral administration or by parenteral administration.

### Embodiment 18

The method of any one of Embodiments 1-15, wherein the method comprises administering the pharmaceutical composition by intramuscular administration or by subcutaneous administration.

### Embodiment 19

The method of any one of Embodiments 1-18, wherein the pharmaceutical composition further comprises a poly(lactide-co-glycolide) copolymer.

### Embodiment 20

The method of Embodiment 19, wherein the poly(lactide-co-glycolide) copolymer is a 50:50 to 95:5 poly(lactide-co-glycolide) copolymer.

### Embodiment 21

The method of any one of Embodiments 1-15 and 17-20, wherein the pharmaceutical composition further comprises N-methyl-2-pyrrolidone.

### Embodiment 22

The method of Embodiment 19 or 20, wherein the poly(lactide-co-glycolide) copolymer is in the form of microspheres.

### Embodiment 23

The method of any one of Embodiments 1-22, wherein the pharmaceutical composition comprises risperidone or the pharmaceutically acceptable salt thereof.

### Embodiment 24

The method of any one of Embodiments 1-22, wherein the pharmaceutical composition comprises risperidone in the form of a free base.

### Embodiment 25

The method of any one of Embodiments 1-22, wherein the pharmaceutical composition comprises risperidone in the form of the pharmaceutically acceptable salt.

### Embodiment 26

The method of any one of Embodiments 1-22, wherein the pharmaceutical composition comprises paliperidone in the form of a free base.

### Embodiment 27

The method of any one of Embodiments 1-22, wherein the pharmaceutical composition comprises paliperidone in the form of a pharmaceutically acceptable salt.

### Embodiment 28

The method of any one of Embodiments 1-25, wherein the pharmaceutical composition comprises about 5 wt % to about 25 wt % risperidone base: about 25 wt % to about 50 wt % of a poly(lactide-co-glycolide)copolymer; and about 35 wt % to about 60 wt % N-methyl-2-pyrrolidone.

### Embodiment 29

The method of any one of Embodiments 1-25, wherein the pharmaceutical composition comprises about 10 wt % to about 20 wt % risperidone base; about 35 wt % to about 45 wt % of a 50:50 to 90:10 poly(lactide-co-glycolide)copolymer; and about 40 wt % to about 50 wt % N-methyl-2-pyrrolidone.

### Embodiment 30A

The method of any one of Embodiments 1-25, wherein the pharmaceutical composition comprises about 15 wt % risperidone base; about 37 wt % of an 80:20 poly(lactide-co-glycolide)copolymer having a number average molecular weight from about 20,000 Daltons to about 30,000 Daltons; and about 47 wt % N-methyl-2-pyrrolidone.

### Embodiment 30B

The method of any one of Embodiments 1-25, wherein the pharmaceutical composition comprises about 15 wt % risperidone base; about 38 wt % of an 80:20 poly(lactide-co-glycolide)copolymer having a number average molecular weight from about 20,000 Daltons to about 30,000 Daltons; and about 47 wt % N-methyl-2-pyrrolidone.

### Embodiment 31

A method for treating schizophrenia in a human having an MC4R gene polymorphism, an HTR2C gene polymorphism, an HTR2A gene polymorphism, or a combination thereof comprising subcutaneously administering once per month a pharmaceutical composition comprising: a therapeutically effective amount of risperidone base, a poly(lactide-co-glycolide)copolymer, and N-methyl-2-pyrrolidone to the human having the MC4R gene polymorphism, the HTR2C gene polymorphism, the HTR2A gene polymorphism, or the combination thereof, to treat the schizophrenia.

### Embodiment 32

The method of Embodiment 31, wherein the human has a TT genotype, a CC genotype, or a CT/TT genotype.

### Embodiment 33

The method of Embodiment 32, wherein the human has the MC4R gene polymorphism and the TT genotype.

### Embodiment 34

The method of Embodiment 32, wherein the human has the MC4R gene polymorphism and the CC genotype.

### Embodiment 35

The method of Embodiment 32, wherein the human has the MC4R gene polymorphism and the CT/TT genotype.

### Embodiment 36

The method of Embodiment 32, wherein the human has the HTR2C gene polymorphism and the TT genotype.

### Embodiment 37

The method of Embodiment 32, wherein the human has the HTR2C gene polymorphism and the CC genotype.

### Embodiment 38

The method of Embodiment 32, wherein the human has the HTR2C gene polymorphism and the CT/TT genotype.

### Embodiment 39

The method of Embodiment 32, wherein the human has the HTR2A gene polymorphism and the TT genotype.

### Embodiment 40

The method of Embodiment 32, wherein the human has HTR2A gene polymorphism and the CC genotype.

### Embodiment 41

The method of Embodiment 32, wherein the human has the HTR2A gene polymorphism and the CT/TT genotype.

### Embodiment 42A

The method of any one of Embodiments 31-35, wherein the MC4R gene polymorphism is a single nucleotide polymorphism.

### Embodiment 42B

The method of Embodiment 42A, wherein the single nucleotide polymorphism is an rs17782313 single nucleotide polymorphism.

### Embodiment 43A

The method of any one of Embodiments 31, 32, and 36-38, wherein the HTR2C gene polymorphism is a single nucleotide polymorphism.

### Embodiment 43B

The method of Embodiment 43A, wherein the single nucleotide polymorphism is an rs3813929 single nucleotide polymorphism.

### Embodiment 44A

The method of any one of Embodiments 31, 32, and 39-41, wherein the HTR2A gene polymorphism is a single nucleotide polymorphism.

### Embodiment 44B

The method of Embodiment 44A, wherein the single nucleotide polymorphism is an rs6313 single nucleotide polymorphism.

### Embodiment 45

The method of any one of Embodiments 31-44, wherein the human is an African-American human.

### Embodiment 46A

The method of any one of Embodiments 31-45, wherein the schizophrenia is acute schizophrenia.

### Embodiment 46B

The method any one of Embodiments 31-45, wherein the schizophrenia is an acute psychotic state.

### Embodiment 46C

The method of any one of Embodiments 31-45, wherein the schizophrenia is a relapse with acute schizophrenic symptoms.

### Embodiment 47

The method of any one of Embodiments 31-46, wherein the pharmaceutical composition comprises from about 60 grams to about 150 grams of risperidone base.

### Embodiment 48

The method of any one of Embodiments 31-47, wherein the pharmaceutical composition comprises about 5 wt % to about 25 wt % risperidone base; about 25 wt % to about 50 wt % of a poly(lactide-co-glycolide)copolymer; and about 35 wt % to about 60 wt % N-methyl-2-pyrrolidone.

### Embodiment 49

The method of any one of Embodiments 31-47, wherein the pharmaceutical composition comprises about 10 wt % to about 20 wt % risperidone base; about 35 wt % to about 45 wt % of a 50:50 to 90:10 poly(lactide-co-glycolide)copolymer; and about 40 wt % to about 50 wt % N-methyl-2-pyrrolidone.

### Embodiment 50A

The method of any one of Embodiments 31-47, wherein the pharmaceutical composition comprises about 15 wt % risperidone base; about 37 wt % of an 80:20 poly(lactide-co-glycolide)copolymer having a number average molecular weight from about 20,000 Daltons to about 30,000 Daltons; and about 47 wt % N-methyl-2-pyrrolidone.

### Embodiment 50B

The method of any one of Embodiments 31-47, wherein the pharmaceutical composition comprises about 15 wt % risperidone base; about 38 wt % of an 80:20 poly(lactide-co-glycolide)copolymer having a number average molecular weight from about 20,000 Daltons to about 30,000 Daltons; and about 47 wt % N-methyl-2-pyrrolidone.

### Embodiment 51

A method for treating a psychiatric disease in a human having a TT genotype, a CC genotype, or a CT/TT genotype comprising administering a pharmaceutical composition comprising a therapeutically effective amount of (i) risperidone or a pharmaceutically acceptable salt thereof or (ii) paliperidone or a pharmaceutically acceptable salt thereof, to the human having the TT genotype, the CC genotype, or the CT/TT genotype to treat the psychiatric disease.

### Embodiment 52

The method of Embodiment 51, wherein the psychiatric disease is schizophrenia.

### Embodiment 53A

The method of Embodiment 52, wherein the schizophrenia is acute schizophrenia.

### Embodiment 53B

The method of Embodiment 52, wherein the schizophrenia is an acute psychotic state.

### Embodiment 53C

The method of Embodiment 52, wherein the schizophrenia is a relapse with acute schizophrenic symptoms.

### Embodiment 54

The method of Embodiment 51, wherein the psychiatric disease is bipolar disorder.

### Embodiment 55

The method of Embodiment 51, wherein the psychiatric disease is autism.

### Embodiment 56

The method of any one of Embodiments 51-55, wherein the human is an African-American human.

### Embodiment 57

The method of any one of Embodiments 51-56, wherein the pharmaceutical composition is an oral tablet or an oral capsule.

### Embodiment 58

The method of any one of Embodiments 51-56, wherein the method comprises administering the pharmaceutical composition to the human by oral administration or by parenteral administration.

### Embodiment 59

The method of any one of Embodiments 51-56, wherein the method comprises administering the pharmaceutical composition by intramuscular administration.

### Embodiment 60

The method of any one of Embodiments 51-56, wherein the method comprises administering the pharmaceutical composition by subcutaneous administration.

### Embodiment 61

The method of Embodiment 60, wherein the poly(lactide-co-glycolide) copolymer is a 50:50 to 95:5 poly(lactide-co-glycolide) copolymer.

### Embodiment 62

The method of any one of Embodiments 51-56, wherein the pharmaceutical composition further comprises N-methyl-2-pyrrolidone.

### Embodiment 63

The method of Embodiment 60 or 61, wherein the poly(lactide-co-glycolide) copolymer is in the form of microspheres.

### Embodiment 64

The method of any one of Embodiments 51-63, wherein the pharmaceutical composition comprises risperidone or the pharmaceutically acceptable salt thereof.

### Embodiment 65

The method of any one of Embodiments 51-63, wherein the pharmaceutical composition comprises risperidone in the form of a free base.

### Embodiment 66

The method of any one of Embodiments 51-63, wherein the pharmaceutical composition comprises risperidone in the form of the pharmaceutically acceptable salt.

### Embodiment 67

The method of any one of Embodiments 51-63, wherein the pharmaceutical composition comprises paliperidone or the pharmaceutically acceptable salt thereof.

### Embodiment 68

The method of any one of Embodiments 51-63, wherein the pharmaceutical composition comprises paliperidone in the form of a free base.

### Embodiment 69

The method of any one of Embodiments 51-63, wherein the pharmaceutical composition comprises paliperidone in the form of a pharmaceutically acceptable salt.

### Embodiment 70

The method of any one of Embodiments 51-56, 58, and 59, wherein the pharmaceutical composition comprises about 5 wt % to about 25 wt % risperidone base, about 25 wt % to about 50 wt % of a poly(lactide-co-glycolide)copolymer; and about 35 wt % to about 60 wt % N-methyl-2-pyrrolidone.

### Embodiment 71

The method of any one of Embodiments 51-56, 58, and 59, wherein the pharmaceutical composition comprises about 10 wt % to about 20 wt % risperidone base; about 35 wt % to about 45 wt % of a 50:50 to 90:10 poly(lactide-co-glycolide)copolymer; and about 40 wt/o to about 50 wt % N-methyl-2-pyrrolidone.

### Embodiment 72A

The method of any one of Embodiments 51-56, 58, and 59, wherein the pharmaceutical composition comprises about 15 wt % risperidone base; about 37 wt % of an 80:20 poly(lactide-co-glycolide)copolymer having a number average molecular weight from about 20,000 Daltons to about 30,000 Daltons; and about 47 wt % N-methyl-2-pyrrolidone.

### Embodiment 72B

The method of any one of Embodiments 51-56, 58, and 59, wherein the pharmaceutical composition comprises about 15 wt % risperidone base; about 38 wt % of an 80:20 poly(lactide-co-glycolide)copolymer having a number average molecular weight from about 20,000 Daltons to about 30,000 Daltons; and about 47 wt % N-methyl-2-pyrrolidone.

### Embodiment 73

A method for treating a psychiatric disease in a human having a single nucleotide polymorphism in the MC4R gene, a single nucleotide polymorphism in the HTR2C gene, a single nucleotide polymorphism in the HTR2A gene, or a combination thereof, comprising administering to the human a therapeutically effective amount of an antipsychotic drug to treat the psychiatric disease.

### Embodiment 74

The method of Embodiment 73, wherein the psychiatric disease is schizophrenia.

### Embodiment 75

The method of Embodiment 73 or Embodiment 74, wherein the human has a CC genotype, a TT genotype, or a CT/TT genotype.

### Embodiment 76

The method of Embodiment 73 or Embodiment 74, wherein the human has a CC genotype, a TT genotype, or a CT/TT genotype.

### Embodiment 77

The method of any one of Embodiments 73-76, wherein the antipsychotic drug is clozapine or a pharmaceutically acceptable salt thereof:

### Embodiment 78

The method of any one of Embodiments 73-76, wherein the antipsychotic drug is loxapine or a pharmaceutically acceptable salt thereof.

### Embodiment 79

The method of any one of Embodiments 73-76, wherein the antipsychotic drug is olanzapine or a pharmaceutically acceptable salt thereof.

### Embodiment 80

The method of any one of Embodiments 73-76, wherein the antipsychotic drug is thioridazine or a pharmaceutically acceptable salt thereof.

### Embodiment 81

The method of any one of Embodiments 73-76, wherein the antipsychotic drug is perphenazine or a pharmaceutically acceptable salt thereof.

### Embodiment 82

The method of any one of Embodiments 73-76, wherein the antipsychotic drug is aripiprazole or a pharmaceutically acceptable salt thereof.

### Embodiment 83

The method of any one of Embodiments 73-76, wherein the antipsychotic drug is iloperidone or a pharmaceutically acceptable salt thereof.

### Embodiment 84

The method of any one of Embodiments 73-76, wherein the antipsychotic drug is ziprasidone or a pharmaceutically acceptable salt thereof.

### Embodiment 85

The method of any one of Embodiments 73-76, wherein the antipsychotic drug is lurasidone or a pharmaceutically acceptable salt thereof.

### Embodiment 86

The method of any one of Embodiments 73-76, wherein the antipsychotic drug is molindone or a pharmaceutically acceptable salt thereof.

### Embodiment 87

The method of any one of Embodiments 73-76, wherein the antipsychotic drug is asenapine or a pharmaceutically acceptable salt thereof.

### Embodiment 88

The method of any one of Embodiments 73-76, wherein the antipsychotic drug is mesoridazine or a pharmaceutically acceptable salt thereof.

### Embodiment 89

The method of any one of Embodiments 73-76, wherein the antipsychotic drug is quetiapine or a pharmaceutically acceptable salt thereof.

### Embodiment 90

The method of any one of Embodiments 73-76, wherein the antipsychotic drug is trifluoperazine or a pharmaceutically acceptable salt thereof.

### Embodiment 91

A method for treating schizophrenia in a human in need thereof comprising administering a therapeutically effective amount of risperidone or a pharmaceutically acceptable salt thereof to the human to treat the schizophrenia, wherein the human has a CC/TT genotype and an rs17782313 single nucleotide polymorphism in the MC4R gene.

### Embodiment 92

A method for treating schizophrenia in a human in need thereof comprising administering a therapeutically effective amount of paliperidone or a pharmaceutically acceptable salt thereof to the human to treat the schizophrenia, wherein the human has a CC/TT genotype and an rs17782313 single nucleotide polymorphism in the MC4R gene.

### Embodiment 93

A method for treating schizophrenia in a human in need thereof comprising administering a therapeutically effective amount of risperidone or a pharmaceutically acceptable salt thereof to the human to treat the schizophrenia, wherein the human has a CC genotype and an rs3813929 single nucleotide polymorphism in the HTR2C gene.

### Embodiment 94

A method for treating schizophrenia in a human in need thereof comprising administering a therapeutically effective amount of paliperidone or a pharmaceutically acceptable salt thereof to the human to treat the schizophrenia, wherein the human has a CC genotype and an rs3813929 single nucleotide polymorphism in the HTR2C gene.

### Embodiment 95

A method for treating schizophrenia in a human in need thereof comprising administering a therapeutically effective amount of risperidone or a pharmaceutically acceptable salt thereof to the human to treat the schizophrenia, wherein the human has a CC genotype and an rs6313 single nucleotide polymorphism in the HTR2A gene.

### Embodiment 96

A method for treating schizophrenia in a human in need thereof comprising administering a therapeutically effective amount of paliperidone or a pharmaceutically acceptable salt thereof to the human to treat the schizophrenia, wherein the human has a CC genotype and an rs6313 single nucleotide polymorphism in the HTR2A gene.

### Embodiment 97

A method for treating schizophrenia in a human in need thereof comprising administering a therapeutically effective amount of risperidone or a pharmaceutically acceptable salt thereof to the human to treat the schizophrenia, wherein the human has a CC/TT genotype and an rs6313 single nucleotide polymorphism in the HTR2A gene.

### Embodiment 98

A method for treating schizophrenia in a human in need thereof comprising administering a therapeutically effective amount of paliperidone or a pharmaceutically acceptable salt thereof to the human to treat the schizophrenia, wherein the human has a CC/TT genotype and an rs6313 single nucleotide polymorphism in the HTR2A gene.

### Embodiment 99

The method of any one of Embodiments 1-15, 18, 51-56, 59, 60, 91, 93, 95, and 97, wherein the composition is an injectable depot formulation containing risperidone in the form of a base or a pharmaceutically acceptable salt; a poly(lactide-co-glycolide) copolymer having a number average molecular weight from about 1000 Daltons to about 5000 Daltons, and an organic solvent selected from the group consisting of benzyl alcohol, benzyl benzoate, ethyl benzoate, N-methyl-2-pyrrolidone, or a combination of two thereof; wherein the risperidone has a particle size distribution such that at least 75% of the particles have a size ranging from about 1 micron to about 40 microns (or less than 38 microns) when measured by a laser light scattering/diffraction technique, such as the Fraunhofer diffraction theory.

### Embodiment 100

The method of any one of Embodiments 1-15, 18, 51-56, 59, 60, 92, 94, 96, and 98, wherein the composition is an injectable depot formulation containing paliperidone in the form of a base or a pharmaceutically acceptable salt; a poly(lactide-co-glycolide) copolymer having a number average molecular weight from about 1000 Daltons to about 5000 Daltons, and an organic solvent selected from the group consisting of benzyl alcohol, benzyl benzoate, ethyl benzoate, N-methyl-2-pyrrolidone, or a combination of two thereof; wherein the risperidone has a particle size distribution such that at least 75% of the particles have a size ranging from about 1 micron to about 40 microns (or less than 38 microns) when measured by a laser light scattering/diffraction technique, such as the Fraunhofer diffraction theory.

### Embodiment 101

The method of any one of Embodiments 1-15, 18, 51-56, 59, 60, 91, 93, 95, and 97, wherein the composition is a solid implantable composition containing risperidone and a biodegradable polymer, and wherein the biodegradable polymer is a polylactic acid; a polyglycolic acid; a combination of a polylactic acid and a polyglycolic acid; or a poly(lactide-co-glycolide) copolymer.

### Embodiment 102

The method of any one of Embodiments 1-15, 18, 51-56, 59, 60, 92, 94, 96, and 98, wherein the composition is a solid implantable composition containing paliperidone and a biodegradable polymer, and wherein the biodegradable polymer is a polylactic acid; a polyglycolic acid; a combination of a polylactic acid and a polyglycolic acid; or a poly(lactide-co-glycolide) copolymer.

### Embodiment 103

The method of any one of Embodiments 1-15, 17, 18, 23-27, 31-47, 51-56, 59, 60, 64-69, and 91-98, wherein the composition is Formulation A.

### Embodiment 104

The method of any one of Embodiments 1-15, 17, 18, 23-27, 31-47, 51-56, 59, 60, 64-69, and 91-98, wherein the composition is Formulation B.

### Embodiment 105

The method of any one of Embodiments 1-15, 17, 18, 23-27, 31-47, 51-56, 59, 60, 64-69, and 91-98, wherein the composition is Formulation C.

### Embodiment 106

The method of any one of Embodiments 1-15, 17, 18, 23-27, 31-47, 51-56, 59, 60, 64-69, and 91-98, wherein the composition is Formulation D.

### EXAMPLES

The following examples are for purposes of illustration only and are not intended to limit the spirit or scope of the disclosure or claims.

### Example 1

Formulation C is a sustained-release (28-day) formulation of risperidone for the treatment of schizophrenia, which is designed to be administered by once-monthly subcutaneous injection. Previously, it has been demonstrated that subjects with acute schizophrenia who received a 90 mg dose or a 120 mg dose of Formulation C (i.e., Formulation C containing 90 mg of risperidone, or Formulation C containing 120 mg of risperidone, respectively) showed significant improvement in Positive and Negative Syndrome Scale (PANSS) total score and Clinical Global Impressions severity of Illness (CGI-S) score compared to placebo over an 8-week period. The aim of this study was to investigate the possible association between genetic polymorphisms in serotonin (HTR2A and HTR2C) and melanocortin (MC4R) receptors and schizophrenia in 350 subjects with acute schizophrenia following treatment with two subcutaneous doses of Formulation C (90 mg and 120 mg) for 8 weeks. The demographics of the humans in the study are shown in Table 1.

TABLE 1

| | | | 90 mg | | 120 mg | |
|---|---|---|---|---|---|---|
| | Placebo | | Formulation C | | Formulation C | |
| | n = 112 | | n = 111 | | n = 114 | |
| Characteristic | n | % | n | % | n | % |
| Gender | | | | | | |
| Male | 81 | 72.3 | 93 | 83.8 | 84 | 73.7 |
| Female | 31 | 27.7 | 18 | 16.2 | 30 | 26.3 |
| Race | | | | | | |
| White | 25 | 22.3 | 28 | 25.2 | 30 | 26.3 |
| Black | 84 | 75.0 | 79 | 71.2 | 80 | 70.2 |
| Asian | 1 | 0.9 | 1 | 0.9 | 3 | 2.6 |
| Native Hawaiian or | 1 | 0.9 | 1 | 0.9 | 1 | 0.9 |
| Pacific Islander | | | | | | |
| Other | 1 | 0.9 | 2 | 1.8 | 0 | 0 |
| Ethnicity | | | | | | |
| Hispanic/Latino | 10 | 8.9 | 7 | 6.3 | 9 | 7.9 |
| Not Hispanic or Latino | 101 | 90.2 | 104 | 93.7 | 104 | 91.2 |
| Unknown | 1 | 0.9 | 0 | 0 | 1 | 0.9 |

| | Mean | SD | Mean | SD | Mean | SD |
|---|---|---|---|---|---|---|
| Age at First Schizophrenia | 26.6 | 9.25 | 25.5 | 8.21 | 26.9 | 8.48 |
| Diagnosis (years) | | | | | | |
| PANSS | | | | | | |
| Total Score | 94.1 | 8.89 | 95.5 | 9.23 | 94.9 | 8.09 |
| Positive symptom | 25.4 | 3.31 | 26.0 | 3.36 | 25.9 | 3.42 |
| subscale | | | | | | |
| Negative symptom | 22.6 | 3.79 | 23.5 | 3.68 | 22.6 | 3.96 |
| subscale | | | | | | |
| General psychopathology | 46.2 | 5.49 | 45.9 | 5.94 | 46.5 | 5.15 |
| subscale | | | | | | |
| CGI-S | 4.8 | 0.59 | 4.8 | 0.58 | 4.8 | 0.48 |

Blood samples were collected prior to the first subcutaneous injection of 90 mg or 120 mg Formulation C or placebo on Day 1 (Visit 3). Three SNPs [HTR2C (rs3813929), HTR2A (rs6313), and MC4R (rs17782313)] were genotyped in 350 subjects. Clinical assessments for efficacy included the PANSS total score and CGI-S score performed at screening and baseline visits and during Visits 3 (Day 1, first injection), 5 (Day 14), 6 (Day 29, second injection), 8 (Day 43), and 9 (end of study, Day 57 or termination visit). The correlation between the genotypes and the change from baseline across study days to the end-of-study in PANSS total score and CGI-S score was evaluated. PANSS and CGI-S scores were analyzed using a mixed-effects model for repeated measures (MMRM), with difference from baseline in PANSS total score or CGI-S score as the dependent variable. Comparison of each dose group with placebo was performed using Dunnett's procedure for controlling Type I error at 2.5% (1-sided)

Results: Melanocortin 4 Receptor—MC4R (rs17782313)

Humans with the CT/TT genotype in the MC4R gene had statistically significant improvement in total PANSS and CGI-S scores compared to humans with the CC genotype.

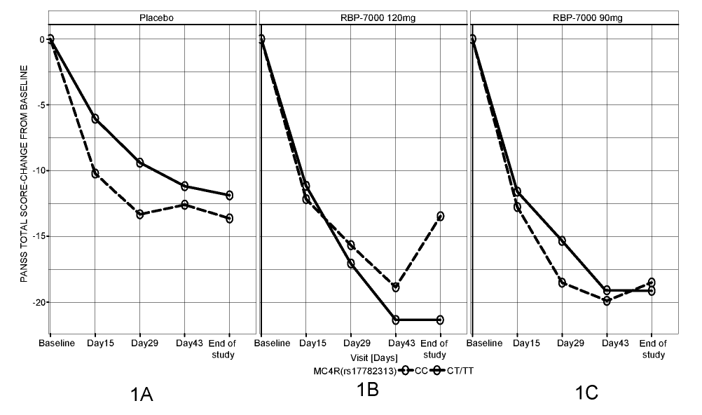

Humans with the CT/TT genotype in the MC4R gene (rs17782313) who received 90 mg of Formulation C (N=101) or 120 mg of Formulation C (N=100) showed significant improvement in the change from baseline in total PANSS scores compared to placebo (N=102) (LSMEAN: −6.498; 95% CI: −10.041 to −2.956; p value: 0.0004 for the 90 mg Formulation C group and LSMEAN: −8.200; 95% CI: −11.748 to −4.652; p value: <0.0001 for the 120 mg Formulation C group). The detailed results are shown in Table 2 and FIG. 1 for the Intent-to-Treat Population.

TABLE 2

| | | | | | LS Mean(SE) | | |
|---|---|---|---|---|---|---|---|
| Receptor | Allele | Treatment | LS Mean(SE) | comparison | Difference | 95% CI | p-value |
| MC4R | CC | Formulation C - | −15.262 | Formulation C - | −3.405 | (−16.386, | 0.5949 |
| (rs17782313) | | 90 mg | (4.4816) | 90 mg - | (6.3267) | 9.577) | |
| | | (N = 9) | | Placebo | | | |
| | CC | Formulation C - | −12.238 | Formulation C - | −0.381 | (−12.405, | 0.9487 |
| | | 120 mg | (3.7751) | 120 mg - | (5.8601) | 11.643) | |
| | | (N = 13) | | Placebo | | | |
| | CC | Placebo | −11.857 | | | | . |
| | | (N = 9) | (4.4657) | | | | |
| | CT/TT | Formulation C - | −15.429 | Formulation C - | −6.498 | (−10.041, | 0.0004 |
| | | 90 mg | (1.2755) | 90 mg - | (1.8000) | −2.956) | |
| | | (N = 101) | | Placebo | | | |
| | CT/TT | Formulation C - | −17.130 | Formulation C - | −8.200 | (−11.748, | <.0001 |
| | | 120 mg | (1.2811) | 120 mg - | (1.8028) | −4.652) | |
| | | (N = 100) | | Placebo | | | |
| | CT/TT | Placebo | −8.930 | | | | . |
| | | (N = 102) | (1.2681) | | | | |

FIG. 1A shows the PANSS total score change from baseline for humans having the MC4R (rs17782313) gene polymorphism receiving a placebo. The lower line represents humans with the CC genotype and the upper line represents humans with the CC/TT genotype. FIG. 1B shows the PANSS total score change from baseline for humans having the MC4R (rs17782313) gene polymorphism receiving a 120 mg dose of Formulation C (referred to in the Figure as RBP-7000). The lower line, with reference to the End of Study, represents humans with a CC/TT genotype and the upper line, with reference to the End of Study, represents humans with the CC genotype. FIG. 1C shows the PANSS total score change from baseline for humans having the MC4R (rs17782313) gene polymorphism receiving a 90 mg dose of Formulation C (referred to in the Figure as RBP-7000). The lower line, with reference to Day 29, represents humans with the CC genotype and the upper line, with reference to Day 29, represents humans with the CC/TT genotype.

With reference to the description of the figures, the term “upper line” refers to the line that is closest to the top of the page, and the term “lower line” refers to the line that is closest to the bottom of the page. Stated alternatively, the term “upper line” refers to the line that is closest to the headers of “Placebo,” “RBP-7000 120 mg,” and “RBP-7000 90 mg,” on the graphs/figures, where all of the headers are closest to the top of the page.

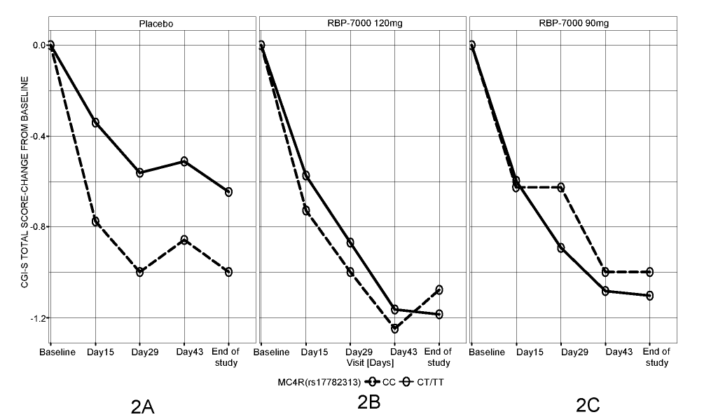

Humans with the CT/TT genotype in the MC4R gene (rs17782313) who received 90 mg Formulation C (N=101) or 120 mg Formulation C (N=100) showed significant improvement in the change from baseline in CGI-S scores compared to placebo (N=102) (LSMEAN: −0.394; 95% CI: −0.586 to −0.202; p value: <0.0001 for the 90 mg Formulation C group and LSMEAN: −0.447; 95% CI: −0.639 to −0.255; p value: <0.0001 for the 120 mg Formulation C group). The detailed results are shown in Table 3 and FIG. 2 for the Intent-to-Treat Population.

TABLE 3

| | | | | | LS Mean(SE) | | |
|---|---|---|---|---|---|---|---|
| Receptor | Allele | Treatment | LS Mean(SE) | comparison | Difference | 95% CI | p-value |
| MC4R | CC | Formulation C - | −0.819 | Formulation C - | 0.076 | (−0.678, | 0.8381 |
| (rs17782313) | | 90 mg | (0.2600) | 90 mg - | (0.3675) | 0.830) | |
| | | (N = 9) | | Placebo | | | |
| | CC | Formulation C - | −0.826 | Formulation C - | 0.069 | (−0.625, | 0.8390 |
| | | 120 mg | (0.2193) | 120 mg - | (0.3387) | 0.764) | |
| | | (N = 13) | | Placebo | | | |
| | CC | Placebo | −0.895 | | | | . |
| | | (N = 9) | (0.2591) | | | | |
| | CT/TT | Formulation C - | −0.878 | Formulation C - | −0.394 | (−0.586, | <.0001 |
| | | 90 mg | (0.0691) | 90 mg - | (0.0974) | −0.202) | |
| | | (N = 101) | | Placebo | | | |
| | CT/TT | Formulation C - | −0.931 | Formulation C - | −0.447 | (−0.639, | <.0001 |
| | | 120 mg | (0.0695) | 120 mg - | (0.0977) | −0.255) | |
| | | (N = 100) | | Placebo | | | |
| | CT/TT | Placebo | −0.484 | | | | . |
| | | (N = 102) | (0.0687) | | | | |

FIG. 2A shows the CGI-S total score change from baseline for humans having the MC4R (rs17782313) gene polymorphism receiving a placebo. The lower line represents humans with the CC genotype and the upper line represents humans with the CC/TT genotype. FIG. 2B shows the CGI-S total score change from baseline for humans having the MC4R (rs17782313) gene polymorphism receiving a 120 mg dose of Formulation C (referred to in the Figure as RBP-7000). The lower line, with reference to the End of Study, represents humans with a CC/TT genotype and the upper line, with reference to the End of Study, represents humans with a CC genotype. FIG. 2C shows the CGI-S total score change from baseline for humans having the MC4R (rs17782313) gene polymorphism receiving a 90 mg dose of Formulation C (referred to in the Figure as RBP-7000). The lower line, with reference to End of Study, represents humans with the CC/TT genotype and the upper line, with reference to End of Study, represents humans with the CC genotype.

Results: Serotonin Subtype 2C Receptor—HTR2C (rs3813929)

Humans with the CC genotype in the HTR2C gene had statistically significant improvement in total PANSS and CGI-S scores following treatment than humans with the CT/TT genotype.

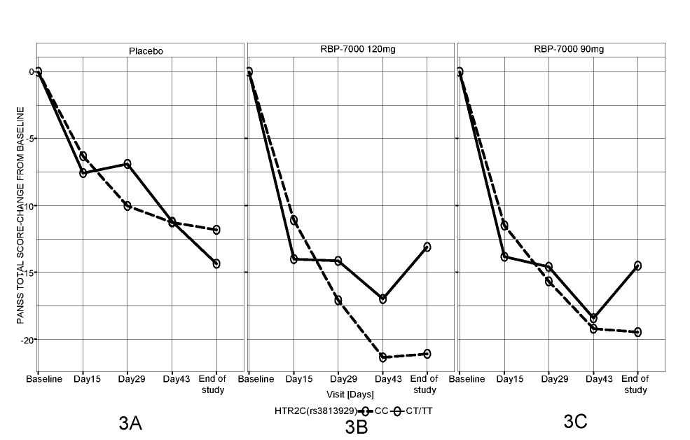

Humans with the CC genotype in the HTR2C gene (rs3813929) who received 90 mg Formulation C (N=102) or 120 mg Formulation C (N=104) showed significant improvement in the change from baseline in total PANSS scores compared to placebo (N=102) (LSMEAN: −6.466; 95% CI: −10.031 to −2.901; p value: 0.0004 for the 90 mg Formulation C group and LSMEAN: −7.733; 95% CI: −11.279 to −4.186; p value: <0.0001 for the 120 mg Formulation C group). The detailed results are shown in Table 4 and FIG. 3 for the Intent-to-Treat Population.

TABLE 4

| | | | | | LS Mean (SE) | | |
|---|---|---|---|---|---|---|---|
| Receptor | Allele | Treatment | LS Mean (SE) | Comparison | Difference | 95% CI | p-value |
| HTR2C | CC | Formulation C - | −15.581 | Formulation C - | −6.466 | (−10.031, | 0.0004 |
| (rs3813929) | | 90 mg | (1.2804) | 90 mg - | (1.8116) | −2.901) | |
| | | (N = 102) | | Placebo | | | |
| | CC | Formulation C - | −16.848 | Formulation C - | −7.733 | (−11.279, | <.0001 |
| | | 120 mg | (1.2679) | 120 mg - | (1.8021) | −4.186) | |
| | | (N = 104) | | Placebo | | | |
| | CC | Placebo | −9.115 | | | | . |
| | | (N = 102) | (1.2802) | | | | |
| | CT/TT | Formulation C - | −14.365 | Formulation C - | −3.911 | (−17.440, | 0.5550 |
| | | 90 mg | (4.7288) | 90 mg - | (6.5239) | 9.619) | |
| | | (N = 8) | | Placebo | | | |
| | CT/TT | Formulation C - | −12.501 | Formulation C - | −2.047 | (−15.166, | 0.7493 |
| | | 120 mg | (4.5025) | 120 mg - | (6.3258) | 11.072) | |
| | | (N = 9) | | Placebo | | | |
| | CT/TT | Placebo | −10.454 | | | | . |
| | | (N = 9) | (4.4408) | | | | |

FIG. 3A shows the PANSS total score change from baseline for humans having the HTR2C gene (rs3813929) gene polymorphism receiving a placebo. The lower line, with reference to Day 29, represents humans with the CC genotype and the upper line, with reference to Day 29, represents humans with the CC/TT genotype. FIG. 3B shows the PANSS total score change from baseline for humans having the HTR2C gene (rs3813929) gene polymorphism receiving a 120 mg dose of Formulation C (referred to in the Figure as RBP-7000). The lower line, with reference to the End of Study, represents humans with a CC genotype and the upper line, with reference to the End of Study, represents humans with a CC/TT genotype. FIG. 3C shows the PANSS total score change from baseline for humans having the HTR2C gene (rs3813929) gene polymorphism receiving a 90 mg dose of Formulation C (referred to in the Figure as RBP-7000). The lower line, with reference to End of Study, represents humans with the CC genotype and the upper line, with reference to End of Study, represents humans with the CC/TT genotype.

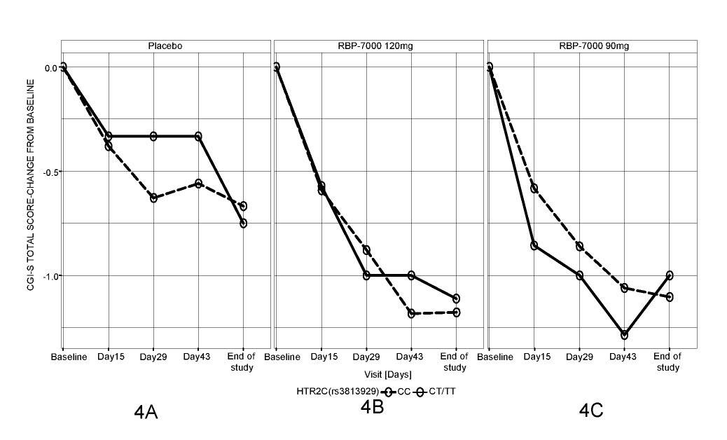

Humans with the CC genotype in the HTR2C gene (rs3813929) who received 90 mg Formulation C (N=102) or 120 mg Formulation C (N=104) showed significant improvement in the change from baseline in CGI-S scores compared to placebo (N=102) (LSMEAN: −0.352; 95% CI: −0.547 to −0.157; p value: 0.0004 for the 90 mg Formulation C group and LSMEAN: −0.411; 95% CI: −0.605 to −0.217; p value: <0.0001 for the 120 mg Formulation C group). The detailed results are shown in Table 5 and FIG. 4 for the Intent-to-Treat Population.

TABLE 5

| Receptor | Allele | Treatment | LS Mean (SE) | Comparison | Difference | 95% CI | p-value |
|---|---|---|---|---|---|---|---|
| HTR2C | CC | Formulation C - | −0.870 | Formulation C- | −0.352 | (−0.547, | 0.0004 |
| (rs3813929) | | 90 mg | (0.0700) | 90 mg- | (0.0990) | −0.157) | |
| | | (N = 102) | | Placebo | | | |
| | CC | Formulation C - | −0.929 | Formulation C- | −0.411 | (−0.605, | <.0001 |
| | | 120 mg | (0.0694) | 120 mg- | (0.0986) | −0.217) | |
| | | (N = 104) | | Placebo | | | |
| | CC | Placebo | −0.519 | | | | . |
| | | (N = 102) | (0.0701) | | | | |
| | CT/TT | Formulation C - | −0.893 | Formulation C- | −0.315 | (−0.998, | 0.3506 |
| | | 90 mg | (0.2333) | 90 mg- | (0.3298) | 0.369) | |
| | | (N = 8) | | Placebo | | | |
| | CT/TT | Formulation C - | −0.796 | Formulation C- | −0.217 | (−0.867, | 0.4953 |
| | | 120 mg | (0.2196) | 120 mg- | (0.3133) | 0.432) | |
| | | (N = 9) | | Placebo | | | |
| | CT/TT | Placebo | −0.579 | | | | . |
| | | (N = 9) | (0.2203) | | | | |

FIG. 4A shows the CGI-S total score change from baseline for humans having the HTR2C (rs3813929) gene polymorphism receiving a placebo. The lower line, with reference to Day 29, represents humans with the CC genotype and the upper line, with reference to Day 29, represents humans with the CC/TT genotype. FIG. 4B shows the CGI-S total score change from baseline for humans having the HTR2C (rs3813929) gene polymorphism receiving a 120 mg dose of Formulation C (referred to in the Figure as RBP-7000). The lower line, with reference to the End of Study, represents humans with a CC genotype and the upper line, with reference to the End of Study, represents humans with a CC/TT genotype. FIG. 4C shows the CGI-S total score change from baseline for humans having the HTR2C (rs3813929) gene polymorphism receiving a 90 mg dose of Formulation C (referred to in the Figure as RBP-7000). The lower line, with reference to End of Study, represents humans with the CC genotype and the upper line, with reference to End of Study, represents humans with the CC/TT genotype.

Results: Serotonin Subtype 2A Receptor—HTR2A (rs6313)

Humans with the CC and CT/TT genotypes in the HTR2A gene had comparable total PANSS and CGI-S scores following treatment.

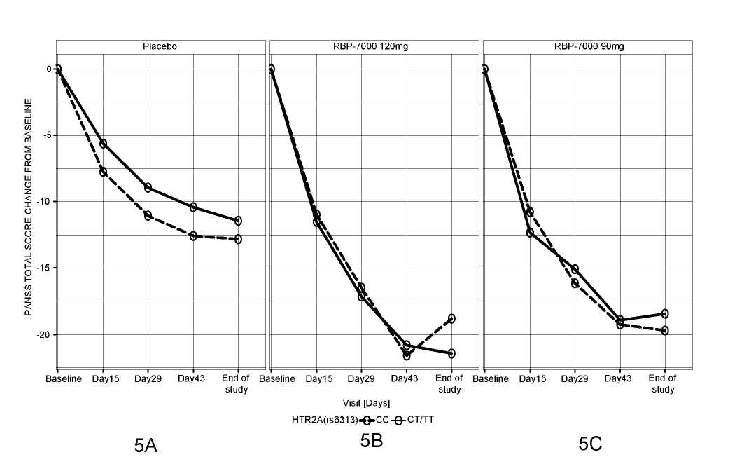

Humans with the CT/TT genotype in the HTR2A gene (rs6313) who received 90 mg Formulation C (N=64) or 120 mg Formulation C showed significant improvement in the change from baseline in total PANSS scores compared to placebo (N=67) (LSMEAN: −6.631; 95% CI: −10.985 to −2.277; p value: 0.0030 for the 90 mg Formulation C group and LSMEAN: −7.967; 95% CI: −12.220 to −3.713; p value: 0.0003 for the 120 mg Formulation C group). The detailed results are shown in Table 6 and FIG. 5 for the Intent-to-Treat Population.

TABLE 6

| Receptor | Allele | Treatment | LS Mean (SE) | Comparison | Difference | 95% CI | p-value |
|---|---|---|---|---|---|---|---|
| HTR2A | CC | Formulation C - | −15.678 | Formulation C - | −5.747 | (−11.438, | 0.0478 |
| (rs6313) | | 90 mg | (2.0173) | 90 mg - | (2.8762) | −0.056) | |
| | | (N = 45) | | Placebo | | | |
| | CC | Formulation C - | −16.275 | Formulation C - | −6.344 | (−12.115, | 0.0315 |
| | | 120 mg | (2.0721) | 120 mg - | (2.9167) | −0.572) | |
| | | (N = 43) | | Placebo | | | |
| | CC | Placebo | −9.931 | | | | . |
| | | (N = 44) | (2.0515) | | | | |
| | CT/TT | Formulation C - | −15.282 | Formulation C - | −6.631 | (−10.985, | 0.0030 |
| | | 90 mg | (1.5791) | 90 mg - | (2.2077) | −2.277) | |
| | | (N = 64) | | Placebo | | | |
| | CT/TT | Formulation C - | −16.617 | Formulation C - | −7.967 | (−12.220, | 0.0003 |
| | | 120 mg | (1.5085) | 120 mg - | (2.1570) | −3.713) | |
| | | (N = 70) | | Placebo | | | |
| | CT/TT | Placebo | −8.650 | | | | . |
| | | (N = 67) | (1.5371) | | | | |

FIG. 5A shows the PANSS total score change from baseline for humans having the HTR2A (rs6313) gene polymorphism receiving a placebo. The lower line represents humans with the CC genotype and the upper line represents humans with the CC/TT genotype. FIG. 3B shows the PANSS total score change from baseline for humans having the HTR2A (rs6313) gene polymorphism receiving a 120 mg dose of Formulation C (referred to in the Figure as RBP-7000). The lower line, with reference to the End of Study, represents humans with a CC/TT genotype and the upper line, with reference to the End of Study, represents humans with a CC genotype. FIG. 3C shows the PANSS total score change from baseline for humans having the HTR2A (rs6313) gene polymorphism receiving a 90 mg dose of Formulation C (referred to in the Figure as RBP-7000). The lower line, with reference to End of Study, represents humans with the CC genotype and the upper line, with reference to End of Study, represents humans with the CC/TT genotype.

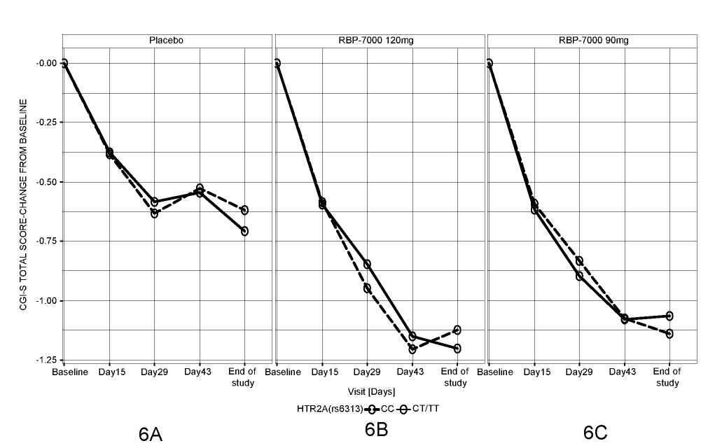

Humans with the CT/TT genotype in the HTR2A gene (rs6313) who received 90 mg Formulation C (N=64) or 120 mg Formulation C (N=70) showed significant improvement in the change from baseline in CGI-S scores compared to placebo (N=67) (LSMEAN: −0.385; 95% CI: −0.624 to −0.145; p value: 0.0018 for the 90 mg Formulation C group and LSMEAN: −0.424; 95% CI: −0.658 to −0.189; p value: 0.0005 for the 120 mg Formulation C group). The detailed results are shown in Table 7 and FIG. 6 for the Intent-to-Treat Population.

TABLE 7

| Receptor | Allele | Treatment | LS Mean (SE) | Comparison | Difference | 95% CI | p-value |
|---|---|---|---|---|---|---|---|
| HTR2A | CC | Formulation C - | −0.870 | Formulation C - | −0.305 | (−0.606, | 0.0471 |
| (rs6313) | | 90 mg | (0.1064) | 90 mg - | (0.1522) | −0.004) | |
| | | (N = 45) | | Placebo | | | |
| | CC | Formulation C - | −0.917 | Formulation C - | −0.352 | (−0.657, | 0.0240 |
| | | 120 mg | (0.1092) | 120 mg - | (0.1541) | −0.047) | |
| | | (N = 43) | | Placebo | | | |
| | CC | Placebo | −0.565 | | | | . |
| | | (N = 44) | (0.1083) | | | | |
| | CT/TT | Formulation C - | −0.878 | Formulation C - | −0.385 | (−0.624, | 0.0018 |
| | | 90 mg | (0.0870) | 90 mg - | (0.1215) | −0.145) | |
| | | (N = 64) | | Placebo | | | |
| | CT/TT | Formulation C - | −0.917 | Formulation C - | −0.424 | (−0.658, | 0.0005 |
| | | 120 mg | (0.0832) | 120 mg - | (0.1188) | −0.189) | |
| | | (N = 70) | | Placebo | | | |
| | CT/TT | Placebo | −0.493 | | | | . |
| | | (N = 67) | (0.0847) | | | | |

FIG. 6A shows the CGI-S total score change from baseline for humans having the HTR2A (rs6313) gene polymorphism receiving a placebo. The lower line, with reference to End of Study, represents humans with the CC/TT genotype and the upper line, with reference to End of Study, represents humans with the CC genotype. FIG. 6B shows the CGI-S total score change from baseline for humans having the HTR2A (rs6313) gene polymorphism receiving a 120 mg dose of Formulation C (referred to in the Figure as RBP-7000). The lower line, with reference to the End of Study, represents humans with a CC/TT genotype and the upper line, with reference to the End of Study, represents humans with a CC genotype. FIG. 6C shows the CGI-S total score change from baseline for humans having the HTR2A (rs6313) gene polymorphism receiving a 90 mg dose of Formulation C (referred to in the Figure as RBP-7000). The lower line, with reference to End of Study, represents humans with the CC genotype and the upper line, with reference to End of Study, represents humans with the CC/TT genotype.

### Summary of Example 1

The results described herein show an association of HTR2C (rs3813929), HTR2A (rs6313), and MC4R (rs17782313) polymorphisms with PANSS and CGI-S scores in humans with acute schizophrenia. This study supports the hypothesis that polymorphisms in the HTR2C (rs3813929), HTR2A (rs6313), and MC4R (rs17782313) receptors may affect individual responses to risperidone, such as risperidone administered as Formulation C.

### Example 2

A long-acting (28-day) subcutaneous formulation of risperidone (Formulation C) was developed for the treatment of schizophrenia to address issues of compliance and non-adherence associated with oral risperidone treatment. Using the medical ranking systems Positive and Negative Syndrome Scale (PANSS) or the Clinical Global Impression-Severity (CGI-S) scale to measure symptom severity in humans, the inventors established an exposure-response relationship between active moiety exposure (risperidone+9-OH-risperidone) and PANSS or CGI-S scores using data from a registration trial. 9-OH-risperidone is also known as paliperidone.

The registration trial consisted of a phase 3 randomized, double-blind, placebo-controlled, multicenter study in 354 humans to evaluate the efficacy, safety and tolerability of Formulation C at two doses, 90 mg and 120 mg. Pharmacokinetic/Pharmacodynamic (PKPD) models were developed using non-linear mixed effects modeling (NONMEM) using the program NONMEM 7.3. These integrated population PKPD models included a joint PK model for risperidone and 9-OH-risperidone together with placebo and drug-effect models. Taken together, these models are necessary to establish the relationship between active moiety exposure and PANSS/CGI-S scores.

Cytochrome P450 2D6 (CYP2D6) is one of the most important enzymes involved in the metabolism of xenobiotics. Subjects classified as CYP2D6 poor and intermediate metabolizers had lower formation rates of 9-OH risperidone (94% and 76% lower respectively) compared to subjects classified as extensive and ultra-rapid CYP2D6 metabolizers. Following Formulation C treatment, the maximum relative decrease in the PANSS score was ˜5% above the placebo effect, half of which could be achieved at a concentration of 4.6 ng/mL of the active moiety. A proportional odds model for CGI-S score related the average concentration to probability of improving scores over time. It is noted that the CYP2D6 phenotype on metabolism of risperidone was the only relevant covariate identified.

Formulation C (also known as RBP-7000) is a long-acting formulation of risperidone administered once monthly via subcutaneous (SC) injection for the treatment of schizophrenia. Formulation C is being developed to address issues of compliance and non-adherence associated with oral risperidone treatment. The safety, tolerability, and pharmacokinetic (PK) profile of Formulation C were evaluated in two Phase 1 single dose studies (a first-in-man study and a single ascending dose study) and in one Phase 2a multiple ascending dose study, all conducted in clinically stable schizophrenic male and female adult subjects. These studies showed that the subcutaneous injection of Formulation C resulted in sustained plasma concentrations of the active moiety over the dosing interval (28 days), and that plasma concentrations close to steady-state were reached after the first subcutaneous injection when transitioning from oral risperidone therapy.

Population PK models of Formulation C were previously developed using data from the single and multiple ascending dose studies. In the single ascending dose study, 45 clinically stable schizophrenic patients with confirmed diagnosis of paranoid, residual or undifferentiated schizophrenia were randomized to receive a single dose of one of the three Formulation C dose levels (60, 90, and 120 mg), with 15 subjects per cohort. The multiple ascending dose study recruited a similar number of patients within the same population as the single ascending dose study and evaluated the effects of three monthly doses (60, 90 and 120 mg) after switching subjects from 2, 3 or 4 mg/day oral risperidone, respectively. Risperidone and 9-OH-risperidone plasma concentration data were well described by a population PK model having the same structural model as the one used for the modeling of the single dose data. Steady-state was reached after the second or third Formulation C subcutaneous injection, but plasma concentrations close to steady state values were obtained right after the first subcutaneous injection when switching from oral risperidone therapy. Data from these two studies were analyzed together using a single population PK model to guide dose selection for phase 3 trials.

The purpose of the present modeling work was to establish an exposure-response relationship between active moiety exposure (risperidone+9-OH-risperidone) and Positive and Negative Syndrome Scale (PANSS) or Clinical Global Impressions severity (CGI-S) scores, using data from a phase 3 registration trial (NCT02109562). The objectives of the population analysis for Formulation C were to (a) characterize the PK profile of Formulation C in schizophrenic patients and to assess the effect of selected covariates on the PK of Formulation C, and (b) characterize the PK/pharmacodynamic (PD) relationships between active moiety plasma concentrations with the PANSS and CGI-S scales.

Methods

A phase 3, randomized, double-blind, placebo-controlled, multicenter study (NCT02109562) was conducted to evaluate the efficacy, safety and tolerability of Formulation C (90 mg and 120 mg) as a treatment in subjects with acute schizophrenia, subjects in an acute psychotic state, or subjects in relapse with acute schizophrenic symptoms, who had a PANSS score of at least 80-120 and a score greater than 4 on at least two of the following four items: hallucinatory behavior, delusions, conceptual disorganization, or suspiciousness/persecution at screening. Subjects were randomly assigned to receive 2 subcutaneous doses of either Formulation C (90 or 120 mg) or placebo over 8 weeks. A total of 538 subjects were screened to enroll in this Phase 3 study. Only 354 subjects passed screening, of which 119 belonged to the placebo group and 116 and 119 belonged to the 90 mg and 120 mg treatment arms, respectively. Furthermore, 17 subjects (7-placebo, 5-90 mg, 5-120 mg) failed to make it to the Intent-to-Treat (ITT) group as they either did not receive at least one dose of Formulation C or did not have at least one assessment post-baseline that facilitates the calculation of change from baseline.

During the screening phase all subjects received 0.25 mg of oral risperidone at Visit 1 (3 to 8 days before double-blind treatment) and a second dose of 0.25 mg oral risperidone 24 hours later to assess their tolerability to risperidone prior to receiving Formulation C. Subjects who passed screening were tapered off their oral anti-psychotic if applicable. Tapering rates for washout medications were at the discretion of the investigator and were determined on an individual basis, with consideration to patient state, dose, and known PK of the medication being tapered, provided the restricted medication was discontinued during Day −8 and Day −1, inclusive. Modifications to subject's pre-existing treatment were not made unless deemed clinically important by the investigator. Upon completion of all study participation requirements, subjects were randomized to one of the three study treatments at Visit 3 (Day 1), corresponding to the start of the 8-week double-blind treatment period: subjects received two subcutaneous injections of Formulation C (90 mg or 120 mg) or placebo at a 28-day interval, on Day 1 and on Day 29.

Pharmacokinetic Sampling

Sparse PK blood samples for analysis of risperidone and 9-OH risperidone plasma levels were collected at specific time points. A pre-dose sample of Day 1 was followed by post-injection samples on Days 1-3, 8-15, 16-22 and on Day 29 just before the second subcutaneous injection of Formulation C. Following the second injection of Formulation C, samples were collected on Days 29-31, 36-42, 43-49 and 55-57.

Pharmacogenetics Sampling

Blood samples for DNA analysis were collected before the first injection of Formulation C on Day 1. These samples were collected to assess how the genetic variation in enzymes and receptors might affect the PK, safety, and efficacy of Formulation C. Genotypes were assessed for cytochrome P450 2D6 (CYP2D6), dopamine D2 receptor (DRD2), serotonin 2C receptor (5-HT2C), serotonin 2A receptor (5-HT2A) and melanocortin 4 receptor (MCR4).

Laboratory Tests

Various laboratory tests were conducted throughout the study duration at specified time points. Tests related to liver function (aspartate aminotransferase—AST; alanine aminotransferase—ALT) and renal function (Creatinine clearance—CRCL) were used as predictors to explore and account for the variability in PK exposure.

Bioanalysis

Plasma concentrations of risperidone and 9-OH risperidone were determined using a validated method of liquid chromatography with tandem mass spectrometry (LC-MS/MS). The analytical methodology was based on the following procedure: 0.05 mL of human plasma containing internal standards (d4-risperidone, d4-9-OH risperidone) was first acidified and then extracted using solid phase extraction. Analysis required evaporation of elute solvent before being reconstituted. An aliquot of the extract was injected onto a Sciex API 5500 LC-MS/MS equipped with an HPLC column. Quantitation was performed using I/x² weighted linear least squares regression analysis generated from fortified plasma calibration standards prepared immediately prior to each run. The method was validated for specificity, linearity, lower limit of quantitation, precision, accuracy, recovery and stability for a range of 0.1 to 100 ng/mL for both risperidone and 9-OH risperidone. The overall precision for both analytes was greater than 11.7%; the overall accuracy was within ±4.4%. The recoveries of both analytes and internal standards were greater than 91%. The established short-term and long-term stability covered the maximum sample storage time.

Efficacy Assessments

The clinical assessments for efficacy included the PANSS and the CGI-S scales. The PANSS scale consists of 30 items, where each item is scored from 1 through 7 (1 indicating the absence of the symptom, and 7 indicating extremely suffering from the symptom). These 30 items are grouped into 3 subscales; positive (7 items), negative (7 items) and general psychopathology (16 items). PANSS scores were collected at baseline on Day 1 and on Days 15+/−1, 29, 43+/−1 and 57. A total of 1571 PANSS assessments were available for analysis from this 8-week study.

CGI-S represents the overall clinical severity of illness for each subject. To perform this assessment, the rater answered the following question: “Considering your total clinical experience with this particular population, how mentally ill is the patient at this time?” Response choices included: 1=normal, not at all ill; 2=borderline mentally ill; 3=mildly ill; 4=moderately ill; 5=markedly ill; 6=severely ill; and 7=among the most extremely ill patients. CGI-S scores were collected at the same times as those for PANSS, i.e., at baseline on Visit 3 (Day 1) and on Days 15+/−1, 29, 43+/−1 and 57. A total of 1998 CGI-S assessments were available for analysis from this 8-week study.

Model Development

A non-linear mixed effects modeling approach to describe the time course of PK, PANSS and CGI-S scores was implemented using the NONMEM 7.3 software. Perl-speaks-NONMEM (PsN, v 4.4.0) was used to operate NONMEM. R software version 3.2.0 (www.r-project.org) was used for graphical inspection of the results. Risperidone and 9-OH risperidone plasma concentrations were used to estimate the parameters of the population PK model, while absolute PANSS scores and CGI-S scores were used for the PD and PK/PD models. The First-Order Conditional Estimation (FOCE) method with or without interaction option in NONMEM was used to estimate population PK and PK/PD model parameters and in the case of CGI-S analysis, LAPLACE method was used. Inter-individual variability (IIV) for the structural model parameters was evaluated assuming lognormal or normal distributions of subject-specific random effects:

Pᵢ=TV×exp^{η}^{ᵢ} or

Pᵢ=TV+ηᵢ

where TV represents the population typical value of the PK and/or PD parameter, and Pᵢ is the value of the parameter for subject i. ηᵢ denotes an individual-specific random effect that distinguishes the value of the i^{th} subject from the TV. The ηᵢ is normally distributed with mean zero and variance ω². The IIV was expressed as percent coefficient of variation (% CV) that was calculated as \sqrt{e^{ω}^{²}−1} for exponential models; for additive models, the CV was calculated as ω/TV.

The intra-individual or residual variability (RUV) describes the variability in residual error, i.e. the variability that remains unexplained and refers to, for example, dosing inaccuracies, analytical assay error, or error in recording sampling times, and structural model misspecifications. A combined proportional and additive error model was used to describe RUV in the plasma concentration, while, an additive term was used to account for the unexplained variability in PANSS score as shown in the following equations:

yᵢⱼ= [symbol] +ϵ₁ᵢⱼ+ [symbol] ×ϵ₂ᵢⱼ for PK Model

yᵢⱼ= [symbol] +ϵᵢⱼ for PANSS PD Model

Where yᵢⱼ is the j^{th} observation in the i^{th} individual, [symbol] is the expectation of yᵢⱼ under the model conditionally to subject i, εᵢⱼ, ε₁ᵢⱼ and ε₂ᵢⱼ are normally-distributed random errors with mean zero and variance σ², σ₁², and σ₂², respectively. In the combined error model σ₁², and σ₂², represent the additive and proportional variance components, respectively.

Model selection was based on comparison of the objective function values (δOFV: 3.84 for one additional estimated parameter, corresponding to a p-value of 0.05) and the goodness-of-fit (GOF) plots. GOF was assessed graphically by evaluation of the agreement between observed and predicted plasma concentrations or PANSS scores, the individual predicted profiles vs. time, the range of individual weighted residuals (IWRES), conditional weighted residuals (CWRES) and expected weighted residuals (EWRES), and the shape of the distribution of these residuals about zero across the range of the predicted concentrations or PANSS scores. The percentage relative standard errors (% RSE) of the parameter estimates and reductions in both IIV and RUV were also used to discriminate between competing models.

Influences of patient and study-specific covariates were evaluated as possible explanatory variables for the variability in the PK or PK/PD model parameters. Covariate analysis was performed in NONMEM using PsN with a step-wise forward additive approach followed by a step-wise backward elimination approach with a p-value of 0.05 and 0.001, respectively. Uncorrelated covariates were included in the model using different functional forms like linear, power and exponential functions.

Pharmacokinetic Model

The population PK of risperidone and 9-OH risperidone after administration of Formulation C was previously described using data from the single and multiple ascending dose studies for Formulation C. Both studies were designed to provide rich plasma concentration-time data that could be used to characterize the PK of Formulation C after single or multiple injections at various doses.

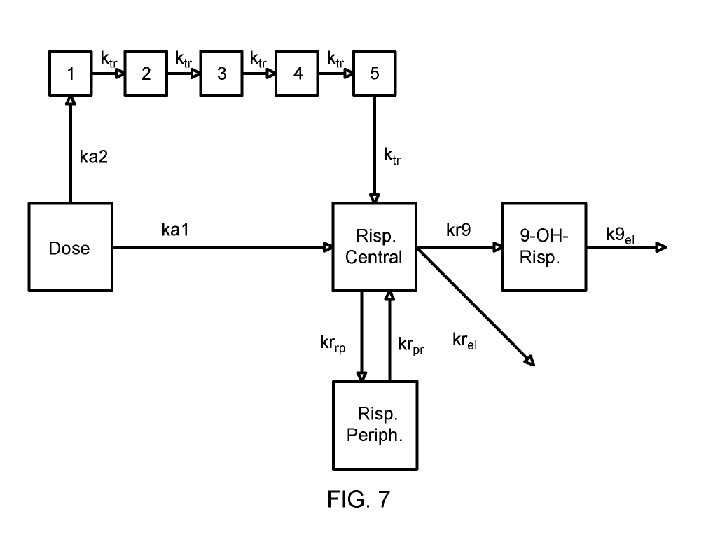

The analysis of the plasma concentration time data from the present phase 3 study was started using the base structural model developed in these two earlier studies (FIG. 7). Briefly, a joint model for risperidone and 9-OH risperidone described the PK profile of Formulation C after subcutaneous injection. A dual absorption process was modeled to account for the double peak plasma concentration-time profiles observed for risperidone and 9-OH-risperidone. This dual absorption process was described by (1) a first-order rate constant (ka1) associated with the first peak; and (2) a transit compartment absorption model, with absorption rate constant ka2 and transit rate constant ktr, associated with the second peak to mimic the slow delivery of risperidone from the formulation. Together with this absorption sub-model, a two-compartment model with two first-order elimination processes described the plasma disposition of risperidone; systemically available risperidone was partly converted into 9-OH risperidone (kr9) and partly eliminated by other routes (krel). The plasma disposition of 9-OH risperidone following Formulation C administration was described by a one-compartment model with first-order elimination (k9el). Since the volume of the 9-OH risperidone was not identifiable, it was set equal to the central volume of the parent compound.

Pharmacodynamic Model of PANSS

PD models usually comprise an underlying disease progression model to capture the changing disease status of schizophrenia with time, and include treatment effects models (placebo and drug) that refer to all the underlying PK and PD processes involved in producing a treatment effect on the time course (t) of disease progression, as shown below:

Disease Status (t)=disease state (t₀)+disease progression (t)+placebo (t)+drug response (t)

where t₀ is the time at the start of the trial. In the present case, it is difficult to separate the disease progression from placebo response due to the episodic nature of schizophrenia and the short duration of the study. Hence, the overall disease progression+placebo response will be referred to as the placebo model.

Placebo Response Model

Several placebo response models were investigated before picking the one that best described the time course of PANSS in the placebo group of the Phase 3 study. Model exploration was conducted using only the placebo data from this study.

Note on Dropouts

Dropouts are common in schizophrenia trials and are in the upper 40-70%. Dropouts, if not accounted for, influence standard errors of estimated model parameters and widen the confidence intervals predicted through simulations. The dropout rates in this phase 3 trial were about 6-9% only across each of the three arms, which is very low when compared to other trials in schizophrenia and were considered unlikely to affect the precision of the parameter estimates, and hence a sub-model to account for dropouts was not considered for this analysis.

PANSS PK/PD—Drug Effect Model

PK/PD modeling relating the time course of PANSS to the drug exposure was performed using a sequential approach. Since the pharmacological profile and potency of 9-OH-risperidone are similar to those of risperidone itself, active moiety plasma concentration was used as a predictor for efficacy. Active moiety [AM] plasma concentrations were calculated as the sum of risperidone and 9-OH-risperidone plasma concentrations, corrected by molecular weight according to the following formula: [AM]=[risperidone]+[9−OH risperidone]×(410/426), where [risperidone] and [9-OH risperidone] are the population PK model-based individual predictions of concentrations.

Sequential (where the placebo response model parameters were initially fixed) and simultaneous analysis of the drug-effect model were explored. Two drug effects models were tested: a linear concentration related slope and an Emax function, both of which affect the change from baseline in PANSS by relating drug exposures.

Pharmacodynamic Model for CGI-S

The overall clinical severity of illness for each subject was rated using the CGI-S ordinal scale with 6 categories. The CGI-S scores were the secondary efficacy endpoint for the Phase 3 study. Due to the low frequency, some levels were consolidated such that the final scores were represented in four categories: (1) Levels 1-3—mildly ill; (2) Level 4—moderately ill; (3) Level 5—markedly ill; and (4) Level 6—severely ill.

As the consolidated CGI-S score is a categorical ordinal variable (taking integer values 3-6) the probability of observing each score was modeled using logistic regression. If Yᵢⱼ denotes the observation in subject i at time tᵢⱼ(j=1 . . . nᵢ) and Yᵢ=(Yᵢ₁, . . . , Yᵢₙᵢ) is the vector of CGI-S scores for subject i, the probability for Yᵢⱼ to be larger than or equal to the score m (m=4, 5 and 6) can be expressed as follows:

G[P(Yᵢⱼ≥m)]=logit(pᵢⱼ)=βₘ+fp+fd+ηᵢ

in which

$$logit ( {p}_{ij} ) = log ( \frac{{p}_{ij}}{1 - {p}_{ij}} )$$

$$and$$

$${p}_{ij} = \frac{exp ( {β}_{m} + fp + fd + {η}_{i} )}{( 1 + exp ( {β}_{m} + fp + fd + {η}_{i} ) )}$$

$${β}_{m} = {\sum }_{l = 4}^{m} {α}_{l}$$

$$fp = TSLP \times {TIME}_{ij}$$

$$fd = CSLP \times {AM}_{ij}$$

where g is the logit function of a probability, pᵢⱼ is short notation for P(Yᵢⱼ≥m). For a M category score, only M−1 intercepts need to be estimated. Here, βₘ decreases with m to account for the ordinal nature of the data; fp represents the placebo effect estimated using placebo data only that is a function of time (TSLP); fd is the drug effect and is a function of active moiety concentration through the parameter CSLP: ηᵢ is the subject-specific random effect introduced on the intercept. The relation is setup such that the covariate relationship is the same for all cumulative categories on the log odds (logits).

The probability of being at a particular score was formulated as:

P(Yᵢⱼ=3)=1−P(Yᵢⱼ≥4)

P(Yᵢⱼ=4)=P(Yᵢⱼ≥4)−P(Yᵢⱼ≥5)

P(Yᵢⱼ=5)=P(Yᵢⱼ≥5)−P(Yᵢⱼ≥6)

P(Yᵢⱼ=6)=P(Yᵢⱼ≥6)

CGI-S PK/PD—Drug Effect Model

Using only data from placebo patients, the formulation listed above was first used to model the CGI-S scores. Subsequently, the treatment arm data was added in a next step where the CGI-S scores were fit as a function of time. The PK/PD model between drug exposure and CGI-S score was established using the plasma concentrations of the active moiety as the predictor.

Covariate Model

Covariate exploration was conducted on each sub-model and the final joint PK/PD model. The inclusion of potential covariates in the final model was based on their clinical relevance and diagnostic plots of empirical Bayes parameter estimates (EBEs) vs. covariate values. The graphical assessment to evaluate covariate relationships looked at the predictions of subject-specific deviations from population mean estimates (η's) for the respective PK or PK/PD parameter from the base model against covariate of interest. Continuous covariates included in this analysis were body weight (BW), age, waist-to-hip ratio (WTH), body mass index (BMI), aspartate aminotransferase (AST), alanine amino transferase (ALT) and creatinine clearance (CRCL). Categorical covariates included gender (male=reference, female), race (African American=reference, Caucasian, Others), ethnicity (Not Hispanic or Latino=reference, Hispanic or Latino), CYP2D6 metabolizer status (Extensive Metabolizers (EM) and Inconclusive=reference, Poor Metabolizers (PM), Intermediate Metabolizers (IM)) and genotype classification for Dopamine D2 (DRD2), Serotonin 2A (5-HT2A), Serotonin 2C (5-HT2C) and Melanocortin 4 (MCR4) receptors. For categorical covariates, covariate effects were added as a proportional component such that the effect estimate represents the % change from the reference typical value of the estimate.

P_{TV}=P_{TV}×(1+slope×(Cov−median_{cov}))

P_{TV}=P_{TV}×((Cov/median_{cov})^{pow})−For continuous covariates

P_{TV}=P_{TV} if Cov==0

P_{TV}=P_{TV}×(1+prop factor) if Cov==1−For categorical covariates

where P2T′ and slope, pow and propfactor are fixed effect parameters representing the population typical value and the covariate effect respectively.

To test the covariate parameter relationship, covariates were added to structural parameters, using the stepwise covariate modeling approach (SCM), as implemented in the PsN software package (5,6). With this technique, different covariate-parameter relationships can be tested in a forward fashion (p<0.05 and δOFV of 3.84) to build up the final full model, which in turn is evaluated in the backward elimination step (p<0.001 and δOFV of 10.8). When a correlation between covariates was found, one of them was omitted on the basis of the prior clinical preference. The resulting final model contains only covariates that meet the pre-defined statistical criteria and show an acceptable precision of related parameter estimates (<50% RSE).

Model Evaluation

A nonparametric bootstrap resampling method was used to evaluate the stability and robustness of the final PK model. Resampling with replacement generated 200 bootstrap data sets and the final population PK model was fitted repeatedly to each of the 200 bootstrap data sets. The median and 95% confidence intervals of parameters obtained from this step were compared with the final parameter estimates. In addition, the prediction-corrected visual predictive check (pcVPC) with 1000 simulated datasets was also performed. In a pcVPC, the variability coming from binning across independent variables is removed by normalizing the observed and simulated dependent variable based on the typical population prediction for the median independent variable in the bin. Results from the VPC were assessed using graphical comparison of the appropriate 90% prediction interval from simulated data and was visually explored in comparison with overlaid observed data from the original dataset. pcVPC's for the PANSS models and categorical VPC's for the CGI-S PD and PK/PD models were used.

Formulation C Pharmacokinetic Analysis

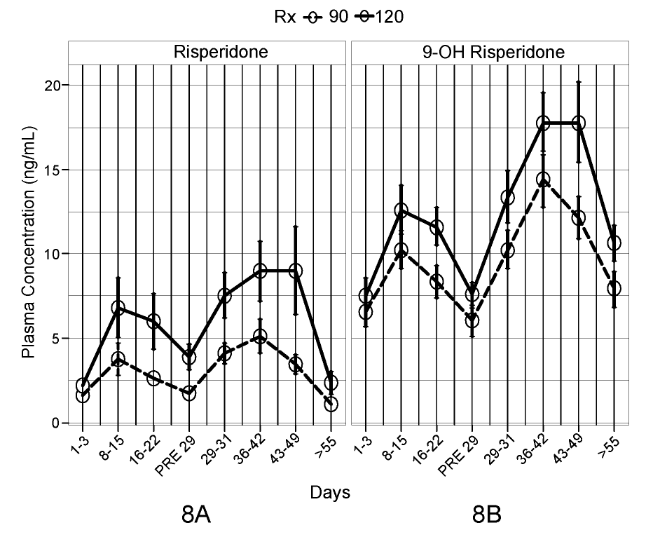

Descriptive statistics in Table 8 provide a summary of the demographics for continuous and categorical covariates. FIG. 8 shows the mean (SD) plasma concentration-time plots for risperidone and 9-OH risperidone in the 90 mg and 120 mg Formulation C groups. The mean profiles in these plots reveal a prolonged absorption phase consistent with the slow delivery of risperidone from the depot formed from Formulation C. Rich PK sampling from the previous Phase 1 studies suggested the presence of two peaks: the first one associated with a rapid delivery from the injection site (first-order absorption process), and the second one associated with the slow delivery from the depot (delayed delivery process described by a transit compartment absorption model). It is difficult to visualize this double peak phenomenon in this sparse design.

TABLE 8

| | | | 90 mg | | 120 mg | |
|---|---|---|---|---|---|---|
| | | | Formulation C | | Formulation C | |
| Characteristic | Placebo group | | group | | group | |
| Total number (n) | 112 | | 111 | | 114 | |
| Age (years) | 42.76 ± 8.65 | | 40.45 ± 9.42 | | 40.41 ± 9.42 | |
| Body Weight (kg) | 92.61 ± 22.86 | | 90.89 ± 18.86 | | 88.54 ± 20.34 | |
| Body Mass Index (kg/m²) | 30.97 ± 7.29 | | 29.59 ± 5.96 | | 29.33 ± 6.73 | |
| Waist-to-hip-ratio | 0.95 ± 0.09 | | 0.95 ± 0.08 | | 0.94 ± 0.07 | |
| Alanine transferase (ALT, U/L) | 29.06 ± 20.09 | | 29.88 ± 21.01 | | 28.54 ± 23.58 | |
| Aspartate transferase (AST, U/L) | 21.65 ± 8.5 | | 23.14 ± 12.13 | | 20.99 ± 8.66 | |
| Creatinine Clearance (CRCL, mL/min) | 122.69 ± 46.91 | | 121.91 ± 32.68 | | 122.62 ± 39.77 | |
| Gender (% male) | 81 | (72.3) | 93 | (83.78) | 84 | (73.6) |
| Ethnicity | | | | | | |
| Not Hispanic or Latino | 101 | (90.1%) | 104 | (93.6%) | 104 | (91.2%) |
| Other | 11 | (9.8%) | 7 | (6.3%) | 10 | (8.7%) |
| Race | | | | | | |
| Black or African American | 84 | (75%) | 79 | (71.1%) | 80 | (70.1%) |
| Other | 28 | (25%) | 32 | (28.8%) | 34 | (29.8%) |
| CYP2D6 Genotype Metabolizer | | | | | | |
| Extensive | 92 | (82.1%) | 98 | (88.2%) | 97 | (85.0%) |
| Intermediate | 8 | (7.1%) | 4 | (3.6%) | 7 | (6.1%) |
| Poor | 3 | (2.6%) | 1 | (0.9%) | 3 | (2.6%) |
| Inconclusive | 8 | (7.1%) | 7 | (6.3%) | 6 | (5.2%) |
| Missing | 1 | (0.9%) | 1 | (0.9%) | 1 | (0.8%) |
| Dopamine D2 receptor (DRD2) | | | | | | |
| genotype | | | | | | |
| GG | 106 | (94.6%) | 108 | (97.2%) | 113 | (99.1%) |
| GC | 5 | (4.4%) | 2 | (1.8%) | 0 | (0%) |
| Missing | 1 | (0.9%) | 1 | (0.9%) | 1 | (0.9) |
| Serotonin 2C receptor (5-HT2C) | | | | | | |
| genotype | | | | | | |
| CC | 102 | (91.0%) | 102 | (91.9%) | 104 | (91.2%) |
| CT | 4 | (3.6%) | 4 | (3.6%) | 3 | (2.6%) |
| TT | 5 | (4.4%) | 4 | (3.6%) | 6 | (5.2%) |
| Missing | 1 | (0.9%) | 1 | (0.9%) | 1 | (0.9%) |
| Serotonin 2A receptor (5-HT2A) | | | | | | |
| genotype | | | | | | |
| CC | 44 | (39.2%) | 45 | (40.5%) | 43 | (37.7%) |
| CT | 55 | (49.1%) | 49 | (44.1%) | 48 | (42.1%) |
| TT | 12 | (10.7%) | 15 | (13.5%) | 22 | (19.2%) |
| Inconclusive | 0 | (0%) | 1 | (0.9%) | 0 | (0%) |
| Missing | 1 | (0.9%) | 1 | (0.9%) | 1 | (0.9%) |
| Melanocortin 4 receptor (MCR4) | | | | | | |
| genotype | | | | | | |
| CC | 9 | (8.0%) | 9 | (8.1%) | 11.4 | (85.0%) |
| TC | 48 | (42.8%) | 55 | (49.5%) | 27.1 | (6.1%) |
| TT | 54 | (48.2%) | 46 | (41.4%) | 60.5 | (2.6%) |
| Missing | 1 | (0.9%) | 1 | (0.9%) | 1 | (0.9%) |

Following inspection of raw data, the 234 subjects from the Formulation C treatment arms contributed to 3154 PK samples that were included in the analysis, 1577 for risperidone and 1577 for 9-OH risperidone. Drug concentrations below the lower limit of quantification (BLQ) were considered as missing. The total number of BLQ was 373 that is approximately 10.3%. However, of the 373 BLQ values, 355 were pre-dose concentrations and the remaining values were post-dose. Considering these extremely small number of BLQ values post-dose (n=18), a decision was made to not include them in the analysis. The pre-dose concentrations above the LLOQ were mainly due to the low dose oral risperidone that was given to all subjects prior to treatment. However, these exposures were insignificant compared to those from the treatment arm (data not shown) and hence were not included in the analysis.

A combined additive and proportional residual error model common to risperidone and 9-OH-risperidone was used in the base model. There were no correlations between the random effects at the subject level. This model provided a reasonable fit of the data and was thus retained for subsequent covariate model development. The exploratory analysis on individual PK parameter predictions (EBEs) vs. covariates identified CYP2D6 phenotype as potential covariate with a strong correlation to the formation of metabolite from the parent. In addition, race was correlated, although to a lesser extent, to both the fast (ka₁) and slow (ka₂) absorption rate constants. So, based on EBE's, CYP2D6 phenotype on metabolite formation and race on ka₁ and ka₂ were selected for testing in NONMEM. BMI was a significant predictor in the previous work, and hence BMI was also taken forward for covariate testing along with CYP2D6 phenotype and race.

Non-parametric bootstrap-based parameter estimates for the final covariate model are presented in Table 9. Stepwise covariate search showed that BMI was not a significant covariate on the absorption rate constants, confirming the lack of correlation in the visual diagnostics of absorption rate constant EBE's vs. BMI. Race was also not significant. Regarding CYP2D6 phenotype, intermediate metabolizers had a 76% lower metabolite formation than extensive/inconclusive metabolizers, while poor metabolizers had a 94% lower metabolite formation.

TABLE 9

| | | Estimate | IIV |
|---|---|---|---|
| Parameter | Description | [95% CI] | [95% CI] |
| ka1 | Rapid abs (h⁻¹) | 0.005 | 41.8 |
| | | [0.004, 0.007] | [35, 50.7] |
| ka2 | Slow abs (h⁻¹) | 0.016 | 32.1 |
| | | [0.01, 0.022] | [0.3, 49.9] |
| krel | Elimination (h⁻¹) | 0.043 | — |
| | | [0.026, 0.074] | |
| V | App. Vol of Risp (L) | 129 | 38.6 |
| | | [96, 158] | [32.4, 44.8] |
| ktr | Transit Rate Constants | 0.023 | 42.3 |
| | (h⁻¹) | [0.021, 0.025] | [31.8, 52.7] |
| krrp | Cen to Per (h⁻¹) | 0.841 | 45.1 |
| | | [0.652, 1.143] | [28.4, 57.1] |
| krpr | Per to Cen (h⁻¹) | 0.006 | 67.9 |
| | | [0.005, 0.008] | [47.9, 81.3] |
| kr9 | Met Formation (h⁻¹) | 0.221 | 49.3 |
| | | [0.179, 0.278] | [37.8, 60.1] |
| K9el | Met Elimination (h⁻¹) | 0.069 | 18.8 |
| | | [0.056, 0.088] | [0.2, 29.5] |
| σ_{add} | Additive RUV | 0.137 | |
| | (ng/mL) | [0.003, 0.214] | |
| σₚᵣₒₚ | Proportional RUV | 0.297 | |
| | | [0.278, 0.321] | |
| CYP2Dt | Effect on Metabolite | −0.76 | |
| intermediate | Formation | [−0.858, −0.567] | |
| CYP2D6 Poor | Effect on Metabolite | −0.942 | |
| | Formation | [−0.968, −0.886] | |

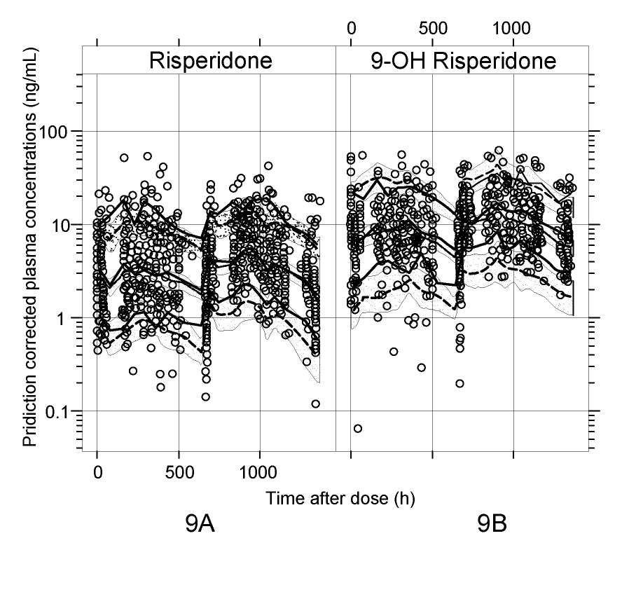

Standard diagnostic plots and representative model fits for the individual PK profiles for risperidone and 9-OH risperidone indicate that the model captured the data very well. Further, a pcVPC stratified by analyte for the final covariate model (FIG. 9) shows that the variability in the data (quantified by 5th and 95th percentiles) was well predicted, with the exception of the upper 95th percentiles which was under- and over-predicted for the parent and metabolite respectively. This was a direct consequence of the presence of some extremely high levels contributed by poor and intermediate metabolizers for CYP2D6.

Pharmacodynamic Model for PANSS

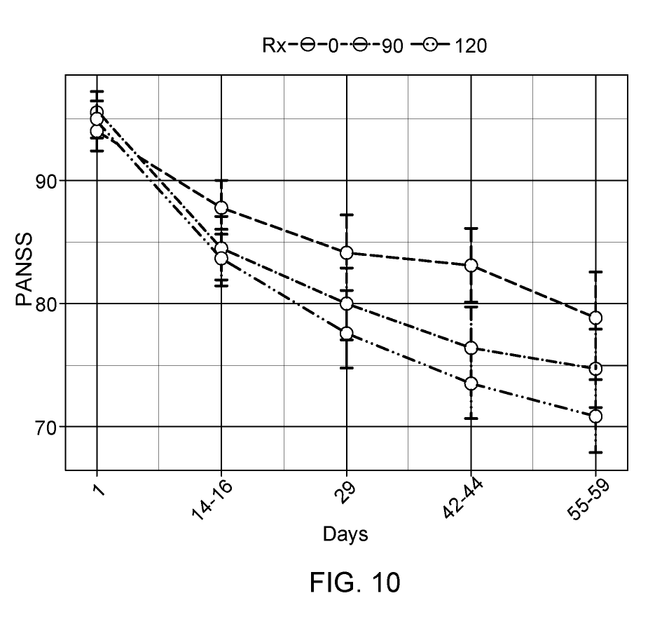

FIG. 10 shows the mean PANSS score over time across the dose groups. There is a clear separation in change from baseline between placebo and treatment arms. There were 527, 526 and 518 PANSS observations in the placebo, 90 mg and 120 mg groups, respectively. It is clear from the plots that on average, PANSS decreased over time in all three treatment groups. Based on the model selection criteria as described in the methods, the Weibull model was chosen over the exponential models even though they had similar OFV, as it provided better individual fits than the exponential model. Further, a linear drift parameter with associated IIV was added to the Weibull model to account for potential individual relapses.

The baseline demographic covariates (age, BMI, WTH, BW, sex, race, ethnicity), CYP2D6 phenotype and the receptor genotypes (5-HT2C, 5-HT2A, MC4R, DRD2) were tested in NONMEM as predictors on this model (Table 8). A stepwise covariate approach found no significant covariates on the placebo model. GOF plots and pcVPC for the placebo model shows that the model captured the data very well.

PK/PD Analysis for PANSS Score

The treatment arm data was then added to the placebo data. Treatment (placebo vs. 90 mg and 120 mg) as a predictor on placebo effect, MDS, was the only expected significant covariate with Formulation C eliciting a maximum change in baseline of about 11.6% as opposed to 5% in the placebo arm. In order to investigate the exposure-response relationship at the subject level, active moiety plasma concentrations were calculated from the individual predictions of risperidone and 9-OH risperidone plasma concentrations as indicated in the methods section. An Eₘₐₓ model best quantified the drug effect, and the parameter estimates of the overall PK/PD model are shown in Table 10. The final exposure response model is depicted by the following equations:

$$DrugEff = \frac{{E}_{ma x} \times AM}{{EC}_{50} + AM}$$

$$PredictedPANSS = BSL \times [ 1 - MDS \times ( 1 - {exp}^{- {( \frac{T}{TPROG} )}^{POW}} ) - DrugEff ] + DRIFT \times T$$

TABLE 10

| | Estimate | BSV | Estimate | BSV |
|---|---|---|---|---|
| | [95% CI] | [95% CI] | [95% CI] | [95% CI] |
| Description | Placebo Model | | Exposure Response Model | |
| BSL - Baseline PANSS | 94.081 | 48.81 | 94.931 | 49.913 |
| | [92.35, 95.71] | [32.07, 69.93] | [93.975, 95.732] | [37.452, 61.409] |
| RUV - Residual Variability | 5.499 | | 5.527 | — |
| | [4.417, 6.555] | | [4.955, 6.051] | |
| MDS - Max Placebo Effect | 0.067 | 0.015 | 0.066 | 0.014 |
| | [0.029, 0.101] | [0.008, 0.022] | [0.037, 0.091] | [0.01, 0.018] |
| TPROG - Time to Reach | 1.953 | — | 1.75 | — |
| Max CFB (weeks) | [1.813, 2.032] | | [1.062, 1.95] | |
| POW - Weibull Shape | 10.064 | — | 2.182 | — |
| Parameter | [2.929, 17.577] | | [1.134, 7.716] | |
| DRIFT - Linear Drift | −0.704 | 2.024 | −1.242 | 1.741 |
| | [−1.124, −0.263] | [0.78, 3.347] | [−1.495, −1.03] | [1.007, 2.349] |
| Eₘₐₓ - Maximum Drug | | | 0.054 | — |
| Effect | | | [0.03, 0.078] | |
| EC 50 - Concentration to | | | 4.604 | — |
| 50% of Eₘₐₓ (ng/mL) | | | [0.19, 21.672] | |

The placebo effect (MDS) for the average schizophrenic patient in this population was estimated to be 0.066 (i.e. the maximum relative decrease in PANSS from the baseline PANSS score was 6.6%). Similarly, the maximum drug effect (E_{Max}) of active moiety (AM) was found to be 0.054 (i.e., the maximum relative decrease in PANSS score from baseline following Formulation C treatment on top of the placebo effect was ˜5.5%). The typical EC₅₀ value for PANSS total score was found to be 4.6 ng/mL. The IIV in EC₅₀ could not be estimated, probably due to the sparseness in this design, and was set to zero. Time when 63.2% of the maximum change from baseline was reached was about 1.7 weeks (˜12 days). The linear drift parameter describes the worsening of the disease in some patients after initial improvement or vice versa. Typical model diagnostics and bootstrap results in Table 10 show that the PK/PD model parameters were well estimated with good precision, except for EC₅₀.

Inspection of Empirical Bayes estimate's relation with covariates did not identify any major trends. Further, a stepwise covariate analysis confirmed that no covariates were significant in this model. Concomitant medications and clinical study site were not tested for their effects, due to the large number of missing data and low number of subjects across the 33 clinical sites to achieve a meaningful effect.

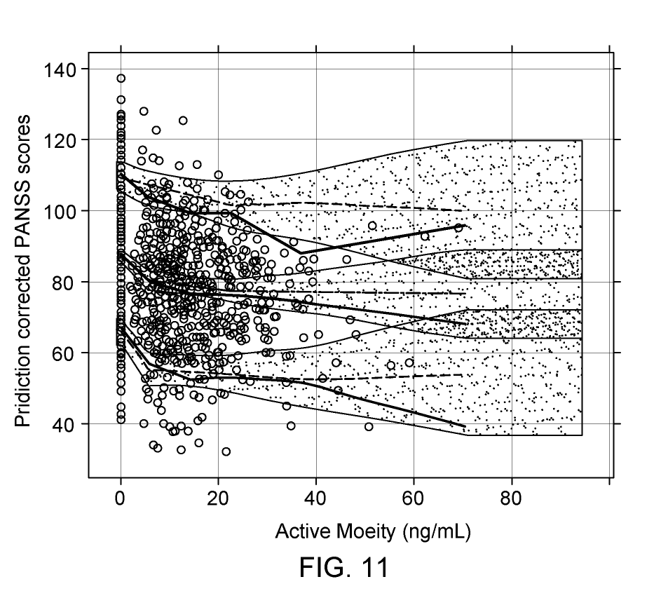

A pcVPC shown in FIG. 11 shows that the model captured both the mean and variability well, although there seemed to be a small under-prediction with regards to change from baseline PANSS. However, the overall trend was well captured.

Pharmacodynamic Model for CGI-S

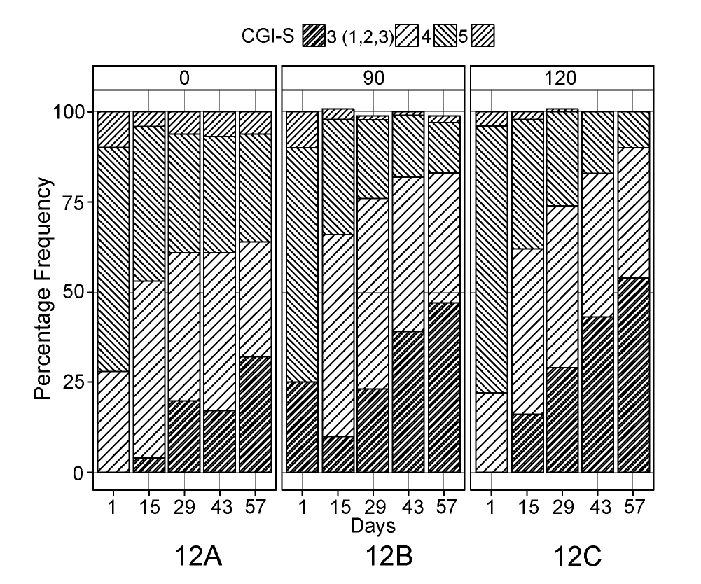

FIG. 12 shows that the proportion of patients with a CGI-S score of 3 (mildly ill) increased over time in all treatment groups, while the proportion of patients with score 5 (markedly ill) decreased. While such trends were expected in Formulation C treatment groups, they were also observed in the placebo group, suggesting the presence of a placebo effect.

There were 518, 516 and 515 CGI-S observations in the placebo, 90 mg and 120 mg treatment group respectively. A placebo model with a time course effect (TSLP×TIMEᵢⱼ) was statistically superior to the base model without the time course (data not shown).

PK/PD Analysis for CGI-S Score

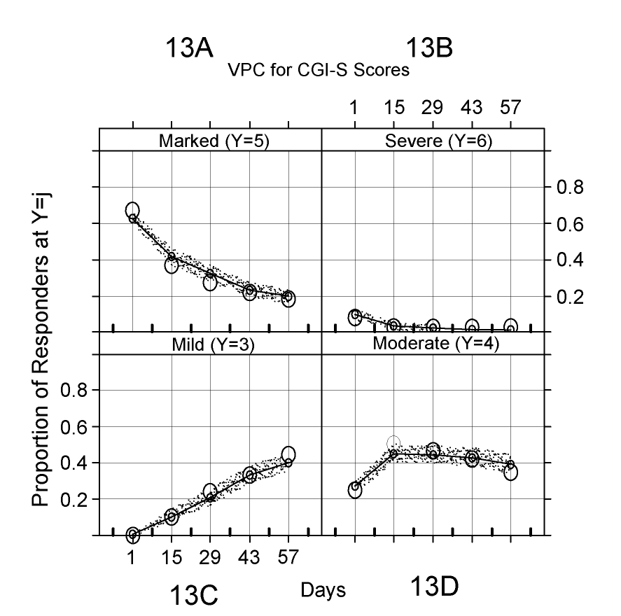

Models with and without a relationship between CGI-S score and Formulation C exposure [dose, active moiety plasma concentration] were tested on top of the placebo model that included a time course on placebo effect. The model that included Formulation C dose as a predictor did not show any significant effect (δ OFV=−1.02); however, the model including active moiety plasma concentration as predictor was significantly better than the model that excluded it (δ OFV=−14.92). No significant improvement was observed in any of the models when the linear relation of concentration (on a logit scale) was replaced by a maximal effect (Eₘₐₓ) model. Significant improvements were observed when IIV was added on the slope for active moiety plasma concentration. FIG. 13 shows a VPC of the time course of the predicted probabilities of different CGI-S scores over time after accounting for active moiety effect. The population estimates of these parameters are listed in Table 11.

TABLE 11

| | Placebo Data | | All Data | | Drug Effect Model | |
|---|---|---|---|---|---|---|
| Description | Estimate | SE | Estimate | SE | Estimate | SE |
| α₄ | 7.86 | 1.08 | 8.46 | 0.68 | 8.51 | 0.68 |
| α₅ | −6.33 | 0.98 | −6.64 | 0.57 | −6.51 | 0.56 |
| α6 | −5.79 | 0.73 | −6.25 | 0.50 | −6.43 | 0.53 |
| TSLP | −0.296 | 0.08 | −0.74 | 0.07 | −0.681 | 0.07 |
| CSLP | — | — | — | — | −0.046 | 0.01 |
| ω₍ₐᵢ₎ | 8.9 | 3.19 | 7.82 | 1.7 | 8.34 | 1.83 |
| ω_{TSLP} | 1.84 | 0.49 | 1.32 | 0.18 | 1.37 | 0.19 |

Discussion

Formulation C is being developed as a long-acting subcutaneous injectable formulation of risperidone given once a month to patients for the treatment of schizophrenia. The long-acting injectable formulation of risperidone currently available in the market (Risperdal® Consta, risperidone by Janssen Pharmaceuticals, Inc.) is given every 2 weeks intramuscularly and requires supplementation with oral risperidone for 3 weeks after the first injection to ensure that adequate therapeutic plasma concentrations are reached. Formulation C has been developed for subcutaneous injection once per month.

The present data originated from a phase 3, placebo-controlled trial comparing 2 doses of Formulation C (90 mg and 120 mg) to placebo in patients with acute schizophrenia. The main objective of this analysis was to establish an exposure-response relationship between the total active moiety concentrations (risperidone+9-OH-risperidone) and PANSS/CGI-S scores. The integrated population PK/PD model developed here was a combination of the following sub-models: (1) a joint PK model for the parent drug and the active metabolite; (2) a PK/PD model for PANSS score; and (3) a PK/PD model for CGI-S score.

The structural PK model was well established from two previous single dose and multiple dose studies where rich data were collected, and hence no effort was made to refine the model for this sparse sampling design. An evaluation of this model provided reasonable fits in most PK profiles from the Phase 3 study and was used for estimating the parameters of this structural model using the Phase 3 study data. Comparison of parameter estimates between the current analysis and previous combined analysis of single dose and multiple dose studies shows consistency, which provides a good external validation to the current PK model. The design of the phase 3 study allowed the collection of PK samples in specific sampling windows that covered the complete time course in the population. Such a design, even with sparse sampling, may have facilitated collection of data that resulted in a population PK model where the parameters were well estimated with reasonable precision.

In the previous single and multiple dose studies, BMI was identified as a significant covariate. This most likely reflects the influence of fat abdominal tissues on the absorption of risperidone which is a lipophilic drug. Hence, in this Phase 3 sparse sampling design, BMI was assessed as a covariate on absorption rate constants, as well as WTH ratio. None of them was identified as significant. The precision of the absorption parameters was good due to the use of sampling time windows in this sparse design. The effect of CYP2D6 phenotype on conversion of risperidone to 9-OH-risperidone has been well established in the literature. In a previous combined analysis by Laffont et al., this effect could not be estimated well due to the low number (n=3) of poor metabolizers. In this study however, even though only 7 poor metabolizers were available in the ITT group, the covariate effect was estimated with statistical significance and the inclusion of the covariate was associated in a 20% drop in IIV on the formation rate constant of the metabolite. Intermediate and poor metabolizers for CYP2D6 had 74% and 96% lower metabolite formation, respectively, than extensive metabolizers. The concentration of the active moiety was however similar across these phenotypes, supporting no need in dose adjustment.

The pcVPC plots shown in FIG. 9 for the covariate PK model well captured the median of the entire time course, but there was marginal underestimation in the upper percentiles. Standard diagnostic plots (not shown) did not show any apparent bias. Overall, the population PK model was considered to adequately describe the data.

The primary endpoint in this phase 3 trial was to assess the efficacy of Formulation C (90 mg and 120 mg) compared with placebo on the symptoms of acute schizophrenia over an 8-week treatment period using the change from baseline to end of treatment in the total PANSS score, which is the sum of all 30 PANSS items. The base placebo model development results for PANSS showed that the Weibull model with a linear slope to account for late relapses, appeared to be adequate to characterize the PANSS data in this 8-week study and was thus selected. This is an empirical placebo model that provided good fits to the data and the pcVPC's showed the good predictive nature of the model. A limitation of such an empirical model is that parameter estimates are based on the study design and duration. A semi-mechanistic or mechanistic model would have been more robust and independent of the study design and duration. However, there are not many semi-mechanistic models due to the complexity and lack of understanding of the disease. This work can be considered for extension to such mechanism based models in the future.

Nevertheless, the empirical base model chosen here to model the placebo PANSS data described the data well with good precision of parameter estimates and excellent simulation properties. The estimates agree well with those reported by Pilla Reddy, with nearly identical numbers for the Weibull parameters, specifically the time to 63.2% of disease progression and the maximum placebo effect. In the present analysis, no covariates were identified on the placebo model to carry forward to the PK/PD model.

The next step was to integrate the PK and the PANSS PD models to establish an exposure-response relationship using a drug-effect model. This integration of critical sub-models such as disease-progression models, placebo-response models, drug-effect models, covariate models and dropout models enables reliable prediction of the outcome of future trials through model-based simulation with consideration of various predictors of the placebo response and dropout. The dropout rate in this phase 3 study was just about 8% and hence a decision was made to not include a dropout sub-model for data description. The parameters estimates of the drug-effect model, Eₘₐₓ and EC₅₀, differ from previous literature mostly because of the choice of independent variable. In this analysis, the active moiety plasma concentration was linked to the effect while other studies used either risperidone concentrations as a predictor, or used a summary statistic of the active moiety PK over the dosing interval. Here, the active moiety concentration was considered more representative of the actual exposure as 9-OH risperidone and risperidone are equally active. The choice of the active moiety plasma concentration in place of any PK summary statistics is explained by the 28-day dosing schedule for Formulation C, i.e., summarizing a 28-day exposure by a few average statistics to correlate to a weekly PD assessment would not be as informative as using the actual concentrations. Moreover, one expects that active moiety concentrations will be correlated with the cumulative average concentrations.

The PK/PD model developed here described the data adequately. The pcVPC's of this integrated model (FIG. 11) show that the PANSS score was predicted well over the range of active moiety concentrations. There were no covariates identified for the PANSS PK/PD model.

The CGI-S scores that range from 1 to 7 had to be consolidated in this study to fewer categories as the number of individual with scores 0 to 2 and 7 was extremely small resulting in just 4 categories of scores. As the data are ordinal in nature, a proportional odds model was selected to model the probability of observing any given score. The placebo model that estimated the baseline probability of being at each score was augmented with a component that explained the time course of the probability that captured the data well. The addition of active moiety plasma concentration as a predictor on the logit of the probabilities significantly improved the model fit and captured the relationship between observed frequency and exposures as seen in FIG. 13. Similar to the PANSS score analysis, no significant covariates were found for the CGI-S placebo model or CGI-S exposure-response model.

The 90 mg and 120 mg doses of Formulation C used in this study were selected as they were expected to provide D2 receptor occupancy levels within the targeted range of 65 to 80%. The results for the Phase 3 study confirm the dose selection with respect to clinical endpoints (PANSS and CGI-S), showing an expected relationship between exposure and response. The PK/PD modeling approach also provides additional insight in evaluating the net effect of the drug (on top of placebo) and relating this effect to the active moiety plasma concentration.

### CONCLUSIONS

Exposure-response analysis of this phase 3 registration trial for Formulation C established a good relationship between the concentrations of active moiety (risperidone+9-OH-risperidone) and PANSS and CGI-S scores. The PK sub-model captured the data well where the parameters were estimated with good precision and consistent with previous knowledge. CYP2D6 phenotype on metabolism of risperidone was the only identified covariate. PANSS and CGI-S models were correlated with the active moiety exposures through a PK/PD model.

### REFERENCES

Baha et al, Metabolism Clinical and Experimental, 59:1156-1163 (2010); Bergstrand et al, AAPS J, 13(2): 143-151 (2011); Czerwensky et al, J Clin Psychopharmacol, 33: 74-79 (2013); Gomeni et al. The Journal of Clinical Pharmacology. 53(10): 1010-1019 (2013); Gobburu, Rev Pharmacol Toxicol, 49:291-301 (2009); Isitt et al, Schizophr Res, 174:126-131 (July 2016); Laffont et al, Clinical Pharmacokinetics, 53:533-543 (2014); Laffont et al, The Journal of Clinical Pharmacology, 55(1):93-103 (January 2015); Love et al, American Journal of Health System Pharmacy, 61:1792-1800 (2004); Malik et al, Der Pharmacia Sinica, 1:74-81 (2010); Nasser et al, J Clin Psychopharmacol, 36(2): 130-140 (April 2016); Penas-Lledo et al, Clin Chem Lab Med, 45:835-838 (2007); Pilla Reddy et al, Schizophr Res, 146(1-3)P:144-152 (2013); Rabinowitz et al, Schizophr Bull, 34(2):286-291 (2008); Wahlby et al, AAPS PharmSci, 4:E27 (2002).

What is claimed is:

1. A method for treating a psychiatric disease in a human having an MC4R gene polymorphism, an HTR2C gene polymorphism, an HTR2A gene polymorphism, or a combination thereof, comprising administering a pharmaceutical composition comprising a therapeutically effective amount of (i) risperidone or a pharmaceutically acceptable salt thereof or (ii) paliperidone or a pharmaceutically acceptable salt thereof, to the human having the MC4R gene polymorphism, the HTR2C gene polymorphism, the HTR2A gene polymorphism, or the combination thereof, to treat the psychiatric disease.

2. The method of claim 1, wherein the human has a TT genotype; a CC genotype; or a CT/TT genotype.

3. The method of claim 1 or 2, wherein the MC4R gene polymorphism is a single nucleotide polymorphism; wherein the HTR2C gene polymorphism is a single nucleotide polymorphism; and wherein the HTR2A gene polymorphism is a single nucleotide polymorphism.

4. The method of claim 1 or 2, wherein the MC4R gene polymorphism is an rs17782313 single nucleotide polymorphism; wherein the HTR2C gene polymorphism is an rs3813929 single nucleotide polymorphism; and wherein the HTR2A gene polymorphism is an rs6313 single nucleotide polymorphism.

5. The method of any one of claims 1-4, wherein the psychiatric disease is schizophrenia, bipolar disorder, or autism.

6. The method of claim 5, wherein the schizophrenia is acute schizophrenia.

7. The method of any one of claims 1-6, wherein the method comprises administering the pharmaceutical composition to the human by oral administration, parenteral administration, or surgical implantation.

8. The method of any one of claims 1-6, wherein the method comprises administering the pharmaceutical composition by intramuscular administration or by subcutaneous administration.

9. The method of any one of claims 1-8, wherein the pharmaceutical composition further comprises a poly(lactide-co-glycolide) copolymer.

10. The method of claim 9, wherein the poly(lactide-co-glycolide) copolymer is a 50:50 to 95:5 poly(lactide-co-glycolide) copolymer.

11. The method of any one of claims 1-10, wherein the pharmaceutical composition further comprises N-methyl-2-pyrrolidone.

12. The method of claim 9 or 10, wherein the poly(lactide-co-glycolide) copolymer is in the form of microspheres.

13. The method of any one of claims 1-8, wherein the pharmaceutical composition comprises about 5 wt % to about 25 wt % risperidone base; about 25 wt/o to about 50 wt % of a poly(lactide-co-glycolide)copolymer; and about 35 wt % to about 60 wt % N-methyl-2-pyrrolidone.

14. The method of any one of claims 1-8, wherein the pharmaceutical composition comprises about 15 wt % risperidone base; about 38 wt % of an 80:20 poly(lactide-co-glycolide)copolymer having a number average molecular weight from about 20,000 Daltons to about 30,000 Daltons; and about 47 wt % N-methyl-2-pyrrolidone.

15. A method for treating schizophrenia in a human having an MC4R gene polymorphism, an HTR2C gene polymorphism, an HTR2A gene polymorphism, or a combination thereof comprising subcutaneously administering once per month a pharmaceutical composition comprising a therapeutically effective amount of risperidone base, a poly(lactide-co-glycolide)copolymer, and N-methyl-2-pyrrolidone, to the human having the MC4R gene polymorphism, the HTR2C gene polymorphism, the HTR2A gene polymorphism, or the combination thereof, to treat the schizophrenia.

16. The method of claim 15, wherein the human has a TT genotype, a CC genotype, or a CT/TT genotype.

17. The method of claim 15 or 16, wherein the MC4R gene polymorphism is a single nucleotide polymorphism; wherein the HTR2C gene polymorphism is a single nucleotide polymorphism; and wherein the HTR2A gene polymorphism is a single nucleotide polymorphism.

18. The method of claim 15 or 16, wherein the MC4R gene polymorphism is an rs17782313 single nucleotide polymorphism; wherein the HTR2C gene polymorphism is an rs3813929 single nucleotide polymorphism; and wherein the HTR2A gene polymorphism is an rs6313 single nucleotide polymorphism.

19. The method of any one of claims 15-18, wherein the schizophrenia is acute schizophrenia.

20. The method of any one of claims 15-18, wherein the pharmaceutical composition comprises from about 60 grams to about 150 grams of risperidone base.

21. The method of any one of claims 15-18, wherein the pharmaceutical composition comprises about 5 wt % to about 25 wt %/o risperidone base; about 25 wt % to about 50 wt %/o of a poly(lactide-co-glycolide)copolymer; and about 35 wt % to about 60 wt % N-methyl-2-pyrrolidone.

22. The method of any one of claims 15-18, wherein the pharmaceutical composition comprises about 15 wt % risperidone base; about 38 wt % of an 80:20 poly(lactide-co-glycolide)copolymer having a number average molecular weight from about 20,000 Daltons to about 30,000 Daltons; and about 47 wt % N-methyl-2-pyrrolidone.

23. A method for treating a psychiatric disease in a human having a TT genotype, a CC genotype, or a CT/TT genotype comprising administering a pharmaceutical composition comprising a therapeutically effective amount of (i) risperidone or a pharmaceutically acceptable salt thereof or (ii) paliperidone or a pharmaceutically acceptable salt thereof, to the human having the TT genotype, the CC genotype, or the CT/TT genotype to treat the psychiatric disease.

24. The method of claim 23, wherein the psychiatric disease is schizophrenia, bipolar disorder, or autism.

25. The method of claim 24, wherein the schizophrenia is acute schizophrenia.

26. The method of any one of claims 23-25, wherein the method comprises administering the pharmaceutical composition to the human by oral administration, by parenteral administration, or by surgical implantation.

27. The method of any one of claims 23-25, wherein the method comprises administering the pharmaceutical composition by intramuscular administration or by subcutaneous administration.

28. The method of any one of claims 23-27, wherein the pharmaceutical composition further comprises a poly(lactide-co-glycolide) copolymer.

29. The method of claim 28, wherein the poly(lactide-co-glycolide) copolymer is a 50:50 to 95:5 poly(lactide-co-glycolide) copolymer.

30. The method of any one of claims 23-29, wherein the pharmaceutical composition further comprises N-methyl-2-pyrrolidone.

31. The method of any one of claims 28-30, wherein the poly(lactide-co-glycolide) copolymer is in the form of microspheres.

32. The method of any one of claims 23-27, wherein the pharmaceutical composition comprises about 5 wt %/o to about 25 wt % risperidone base; about 25 wt % to about 50 wt % of a poly(lactide-co-glycolide)copolymer; and about 35 wt % to about 60 wt % N-methyl-2-pyrrolidone.

33. The method of any one of claims 23-27, wherein the pharmaceutical composition comprises about 15 wt % risperidone base; about 38 wt % of an 80:20 poly(lactide-co-glycolide)copolymer having a number average molecular weight from about 20,000 Daltons to about 30,000 Daltons; and about 47 wt % N-methyl-2-pyrrolidone.

34. A method for treating a psychiatric disease in a human having a polymorphism in the MC4R gene, a polymorphism in the HTR2C gene, a polymorphism in the HTR2A gene, or a combination thereof, comprising administering to the human a therapeutically effective amount of an antipsychotic drug to treat the psychiatric disease.

35. The method of claim 34, wherein the psychiatric disease is schizophrenia.

36. The method of claim 34 or 35, wherein the human has a CC genotype, a TT genotype, or a CT/TT genotype.

37. The method of any one of claims 34-36, wherein the HTR2C gene has a single nucleotide polymorphism; the HTR2A gene has a single nucleotide polymorphism; and the MC4R gene has a single nucleotide polymorphism.

38. The method of any one of claims 34-36, wherein the HTR2C gene has an rs3813929 single nucleotide polymorphism; the HTR2A gene has an rs6313 single nucleotide polymorphism; and the MC4R gene has an rs17782313 single nucleotide polymorphism.

39. The method of any one of claims 34-38, wherein the antipsychotic drug is clozapine or a pharmaceutically acceptable salt thereof; loxapine or a pharmaceutically acceptable salt thereof; olanzapine or a pharmaceutically acceptable salt thereof: thioridazine or a pharmaceutically acceptable salt thereof, perphenazine or a pharmaceutically acceptable salt thereof; aripiprazole or a pharmaceutically acceptable salt thereof; iloperidone or a pharmaceutically acceptable salt thereof; ziprasidone or a pharmaceutically acceptable salt thereof; lurasidone or a pharmaceutically acceptable salt thereof; molindone or a pharmaceutically acceptable salt thereof; asenapine or a pharmaceutically acceptable salt thereof; mesoridazine or a pharmaceutically acceptable salt thereof; quetiapine or a pharmaceutically acceptable salt thereof; or trifluoperazine or a pharmaceutically acceptable salt thereof.
